SEQUENCE LISTING (12 sequences)

SEQ ID NO: 1 - DNA, 24 bp, Artificial Sequence
  PCR primer
ctggaggtggtaatgtgaagattg

SEQ ID NO: 2 - DNA, 20 bp, Artificial Sequence
  PCR primer
tctcagccccgtgatctacc

SEQ ID NO: 3 - DNA, 20 bp, Artificial Sequence
  PCR primer
aaccagcaactccacccttc

SEQ ID NO: 4 - DNA, 20 bp, Artificial Sequence
  PCR primer
cgaattgcccgaacatttgc

SEQ ID NO: 5 - DNA, 20 bp, Artificial Sequence
  PCR primer
ctaacgactatcgccgccaa

SEQ ID NO: 6 - DNA, 20 bp, Artificial Sequence
  PCR primer
caggaatggtgatgcggttt

SEQ ID NO: 7 - DNA, 20 bp, Artificial Sequence
  PCR primer
caacgaaacacttctgggcg

SEQ ID NO: 8 - DNA, 20 bp, Artificial Sequence
  PCR primer
ccagaggcgcataccacatt

SEQ ID NO: 9 - DNA, 20 bp, Artificial Sequence
  PCR primer
gaaccccgaaaggtgtggat

SEQ ID NO: 10 - DNA, 25 bp, Artificial Sequence
  PCR primer
ttccgaatgtgaattagatttgagg

SEQ ID NO: 11 - DNA, 24 bp, Artificial Sequence
  PCR primer
ctctggctcctagcaccatgaaga

SEQ ID NO: 12 - DNA, 26 bp, Artificial Sequence
  PCR primer
gtaaaacgcagctcagtaacagtccg

(19) United States
(12) Patent Application Publication
(10) Pub. No.: US 2024/0279603 A1
(43) Pub. Date: Aug. 22, 2024

(54) METHODS OF PROCESSING ADULT NEURAL CELLS FROM MAMMALS AND ASSAYS THEREOF

(71) Applicant: THE TEXAS A&M UNIVERSITY SYSTEM, College Station, TX (US)

(72) Inventors: Arthur SEFIANI, College Station, TX (US); Cedric G. GEOFFROY, College Station, TX (US)

(21) Appl. No.: 18/571,505

(22) PCT Filed: Jun. 17, 2022
(86) PCT No.: PCT/US22/33972

§ 371 Date: Dec. 18, 2023

Related U.S. Application Data
(60) Provisional application No. 63/212,221, filed on Jun. 18, 2021.

(51) Int. Cl. C12N 5/0793

(52) U.S. Cl. CPC C12N 5/0619; C12N 2500/90; C12N 2501/727; C12N 2501/734; C12N 2509/10

(57) ABSTRACT

The present disclosure provides method of processing neural cells comprising extracting one or more brain components from an animal, dissociating cells from the brain components to form a brain cell composition, purifying the neural cells from the brain cell composition, and culturing the neural cells. Further, the present disclosure provides in vitro assays and related kits utilizing the neural cells, for example in use to screen compounds or therapeutics for neurological diseases or disorders.

### CROSS-REFERENCE TO RELATED APPLICATIONS

This application claims the benefit under 35 USC § 119(e) of U.S. Provisional Application Ser. No. 63/212,221, filed on Jun. 18, 2021, the entire disclosure of which is incorporated herein by reference.

### REFERENCE TO SEQUENCE LISTING SUBMITTED ELECTRONICALLY

The official copy of the sequence listing is submitted concurrently with the specification as a text file via EFS-Web, in compliance with the American Standard Code for Information Interchange (ASCII), with a file name of 364994_ST25(22917599.1).txt, a creation date of Jun. 17, 2022, and a size of 2.41 kb. The sequence listing filed via EFS-Web is part of the specification and is hereby incorporated in its entirety by reference herein.

### BACKGROUND AND SUMMARY OF THE INVENTION

As the median age of the population continues to increase, the prevalence of age-associated neurological disorders such as neurodegenerative disorders and neurotrauma also escalates. For example, from 1990 to 2010, worldwide dementia cases increased by 99.3% and the per capita rate for dementia increased by 53.3%. Likewise, from 1978 to 2005, the percentage of geriatric people with spinal cord injury (SCI) increased by 267%. Further, from 2001 to 2010, the prevalence of traumatic brain injury (TBI) in senior citizens increased by 53.5% while simultaneously decreasing for young adults.

Generally, after the onset of neurodegenerative diseases and neurotrauma, the survival of neurons decreases and escalates pathological and neurobehavioral outcomes. Neurite growth is a vital step in the recovery process from neurotrauma in order to reconnect damaged connections. In the case of SCI, axons extended from cortical neurons are damaged and degenerated and, thus, must be regenerated past the lesion site to restore connections to caudal neurons. Neurodegenerative diseases also induce axonal degeneration which can lead to neuronal death and progression of pathology.

Current therapeutic strategies in neurodegenerative diseases, such as glaucoma and Parkinson's Disease, aim at increasing axon regenerative capacity to mitigate pathology. Aging itself reduces neurite regenerative capacity and increases susceptibility of neurons to death and degeneration which can result in poor outcomes after neurotrauma and increases the prevalence of neurodegenerative diseases. Therefore, there is a growing need to identify and develop novel therapeutic strategies to address neurological disorders in the older population, in particular therapeutics that can increase the survival and neurite regenerative capacity of neurons in older patients.

However, identification of therapeutic compounds to address neurological disorders in the older population is extremely challenging. An efficient way to identify compounds that increase the survival and neurite regenerative capacity of neurons is to perform high content in vitro screenings. Current screens utilize cell lines, embryonic neurons, newborn neurons, and iPSC-derived neurons but are a poor predictor of an older patient population because they do not represent the target neuron population. Indeed, aged neurons have different characteristics relative to neurons from younger individuals. This dichotomy in age between current in vitro assays and desired in vivo settings likely influences the large number of clinical failures for finding new therapeutics for neurological disorders in the older population.

Furthermore, sex-based differences in pharmacological response, pharmacodynamics, and pharmacokinetics are also problematic. For the older patient population, sex-based differences in motor performance and brain characteristics only become more apparent after puberty takes place. Thus, drug screening in embryonic or early postnatal neurons cannot properly account for sex-based differences that may be essential for identifying therapeutics.

Moreover, culturing adult neurons has proven to be challenging. In fact, the few protocols to culture even young adult cortical neurons lead to very inconsistent results including low yields and poor cell viability. This detriment renders testing of a single drug candidate on young adult neurons nearly impossible, let alone testing of candidates on older cortical neurons.

Accordingly, the present disclosure provides methods for processing adult neural cells and in vitro assays utilizing the neural cells. The methods provided herein are capable of providing millions of adult neural cells (including young adult, middle-aged adult, and senior adult cells) that can be cultured and utilized to further treatment of neurological disorders in the older population. For instance, the methods and assays described herein can allow for screening of therapeutic compounds that are able to enhance survival and neurite outgrowth and also determine the demographics for which efficacy is most pronounced. By using adult neural cells instead of embryonic or postnatal neural cells, it may be possible to identify therapeutic compounds to treat neurological disorders in an older population that may not have an effect on the younger neural cells. In this regard, possible drug compounds that may have been prematurely dismissed as false negatives in previous screens of embryonic or postnatal neural cells may be shown to be beneficial in treating adult neural cells. Further, the methods and assays of the present disclosure can provide examination of sex-dependent effects of compounds, an essential variable in drug development. This facet may allow for the development of future age and sex-based personalized medicine.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C show the effects of Laminin coating and media supplementation. Histograms of the average neurite length (FIG. 1A), total neurite outgrowth (FIG. 1B), and number of valid neurons (FIG. 1C) are expressed as percent change relative to the control group containing no B27+ supplement or laminin coating. Primary cortical neurons were isolated from young adult male mice and cultured for 2DIV. 3 wells per condition. * (P<0.05), ** (P<0.01), *** (P<0.001), **** (P<0.0001). Graphs show mean and SEM.

FIGS. 2A-2F show the effects of different digestion enzymes and concentrations. Histograms of the average neurite length (FIG. 2A), total neurite outgrowth (FIG. 2B), and number of valid neurons (FIG. 2C) are expressed as percent change relative to the MACS P&A group. Representative 20× magnification images of primary cortical neurons isolated using the MACS P&A and E (FIG. 2D) and 0.3 mg/mL Papain (FIG. 2E and a 63× magnification in FIG. 2F) dissociation enzymes from young adult male mice and cultured for 2DIV; stained with TUBB3 (Green) and DAPI (Blue). 6 wells per condition. * (P<0.05), ** (P<0.01), *** (P<0.001), **** (P<0.0001). Graphs show mean and SEM. Scale Bar=50 μm (D, E) or 20 μm (F).

FIGS. 3A-3C show the effects of different dissociation protocols, timing, and temperature. Histograms of the average neurite length (FIG. 3A), total neurite outgrowth (FIG. 3B), and number of valid neurons (FIG. 3C) are expressed as percent change relative to the standard gentleMACS Program 37C_ABDK_01 protocol (ABDK (30 minute)). Parentheses represent the length of the protocol whether being ran on the gentleMACS™ Octo Dissociator with Heaters (ABDK) or in a revolving apparatus incubated at 37° C. or 25° C. Primary cortical neurons isolated from young adult male mice and cultured for 2DIV. 6 wells per condition. * (P<0.05), ** (P<0.01), *** (P<0.001), **** (P<0.0001). Graphs show mean and SEM

FIGS. 4A-4I show effects of cell plating density. Linear trends of the average neurite length (FIG. 4A), total neurite outgrowth (FIG. 4B), and number of valid neurons (FIG. 4C) of Vehicle and (S)-H-1152 treated primary cortical neurons isolated from young adult male mice cultured for 2DIV. The X-axis denotes the number of cells plated in each well. Representative 20× magnification images of primary cortical neurons from young adult male mice treated with Vehicle for 2DIV and plated at 1,500 cells/well (FIG. 4D), 3,750 cells/well (FIG. 4E), 5,000 cells/well (FIG. 4F), 7,500 cells/well (FIG. 4G), 10,000 cells/well (FIG. 4H), and 15,000 cells/well (FIG. 4I) stained with TUBB3 (Green) and DAPI (Blue). * (P<0.05), ** (P<0.01), *** (P<0.001), **** (P<0.0001). Simple liner regression conducted to determine goodness of fit and if the slope differs significantly from 0. Linear regression t-test was used to compare the slope of the regression lines. 2 wells per condition. Graphs show mean and SEM. Scale Bar=50 μm.

FIGS. 5A-5C show efficacy of various neuronal supplements. Histograms of the average neurite length (FIG. 5A), total neurite outgrowth (FIG. 5B), and number of valid neurons of primary cortical neurons (FIG. 5C) treated with the respective neuronal supplement isolated from young adult male mice and cultured for 2DIV. Data are expressed as percent change relative to the B27+ supplement cohort. 3 wells per condition. * (P<0.05), ** (P<0.01), *** (P<0.001), **** (P<0.0001). Graphs show mean and SEM.

FIGS. 6A-6C show effects of the drug vehicle DMSO. Linear trends of the average neurite length (FIG. 6A), total neurite outgrowth (FIG. 6B), and number of valid neurons (FIG. 6C) of primary cortical neurons isolated from young adult male mice cultured in various percentages of DMSO for 2DIV. The X-axis denotes the percentage of the neuron media comprising of DMSO. The values are expressed as percent change relative to the 0% DMSO cohort. Simple liner regression conducted to determine fit and if the slope differs significantly from 0. There are 2 wells per condition. Graphs show mean and SEM.

FIGS. 7A-7C show culture purity assessed with RNA expression analysis. Histograms of the mass of RNA collected from each preparation (FIG. 7A), the relative expression of NeuN expressed as 2-ΔCT (NeuN) (FIG. 7B), and the relative expression of NeuN (FIG. 7C) in relation to GFAP and Glast comparing the original Miltenyi protocol or the protocol of Example 1. −ΔCT=−(ΔCT NeuN−(SQRT(ΔCT GFAP2+ΔCT Glast2))). All ΔCT values are calculated as follows: ΔCT Primer=CT Primer (Sample)−CT Primer (Negative Control). There were 3 independent samples per cohort, each analyzed in triplicate. * (P<0.05), ** (P<0.01), *** (P<0.001), **** (P<0.0001). Graphs show mean and SEM.

FIGS. 8A-8H show sex- and age-dependent effects of RO48. Histograms of the average neurite length (FIG. 8A), total neurite outgrowth (FIG. 8B), and number of valid neurons (FIG. 8C) extracted from the vehicle treated group, expressed as percent change relative to the young adult female (Young Female) cohort. * (P<0.05), ** (P<0.01), *** (P<0.001), **** (P<0.0001). Linear trends of the average neurite length (FIG. 8D), total neurite outgrowth (FIG. 8E), and number of valid neurons (FIG. 8F) of primary cortical neurons isolated from young adult male, young adult female, and middle-aged female mice cultured in various concentrations of RO48 for 2DIV. The X-axis denotes the concentration (nM) of RO48 in the media (FIGS. 8D-8F). The values are expressed as percent change relative to the Vehicle treatment group of each cohort. Representative 20× magnification images of Vehicle (FIG. 8G) and 3400 nM RO48 treated primary cortical neurons (FIG. 8H) isolated from middle-aged female mice and cultured for 2DIV and stained with TUBB3 (Green) and DAPI (Blue). Simple liner regression was conducted to determine goodness of fit and if the slope differs significantly from 0. #(P<0.05), ##(P<0.01), ###(P<0.001), ####(P<0.0001). Comparison of the means of neurons treated with various concentrations of RO48 to the Vehicle treatment group of the respective cohort are denoted by * (P<0.05), ** (P<0.01), *** (P<0.001), **** (P<0.0001). All cohorts have equal parts Vehicle in media (0.05% DMSO). 3 wells per condition. Graphs show mean and SEM. Scale Bar=50 μm.

FIGS. 9A-9C show age-dependent effects of 7-epi Paclitaxel. Histograms of the average neurite length (FIG. 9A), total neurite outgrowth (FIG. 9B), and number of valid neurons (FIG. 9C) are expressed as percent change relative to the young adult male cohort with vehicle treatment. Primary cortical neurons isolated from young adult male, young adult female, and middle-aged male mice were cultured in vehicle or 150 nM 7-epi Paclitaxel or 3DIV. All cohorts had equal parts vehicle in media (0.05% DMSO). 4 wells per condition. * (P<0.05), ** (P<0.01), *** (P<0.001), **** (P<0.0001). Graphs show mean and SEM.

FIG. 10 shows the screening platform using primary adult neural cells from mice as used in Example 1. Briefly, the cortex was extracted from mice, dissociated in a dissociator, and followed by the removal of debris and red blood cells. Afterwards, the glial cells were separated from neurons.

FIG. 11 shows an exemplary processing and screening method using neural cells obtained from adult sheep.

FIGS. 12A-12D show results from a screen of cortical neurons obtained from a 2-year old adult sheep brain. The cortical neurons were cultured for 2DIV and stained with secondary antibody Alexa 456 (Goat) only (FIG. 12A), or with TUBB3 primary antibody with Alexa 546 (FIGS. 12B and 12C). Cells were treated with either vehicle control (FIGS. 12A-12B) or with 2.5 μM of RO48 treatment (FIG. 12C). FIG. 12D shows a TUBB3 Western blot is shown against 15 μg of protein from mouse and sheep cortices next to iBright ladder. 2 DIV, 15,000 cells/well. Scale bar=50 μm (white).

### DETAILED DESCRIPTION

Various embodiments of the invention are described herein as follows. In an illustrative aspect, a method of processing neural cells is provided. The method comprises the steps of extracting one or more brain components from an animal, dissociating cells from the brain components to form a brain cell composition, purifying the neural cells from the brain cell composition, and culturing the neural cells.

In an embodiment, the disassociating step comprises enzymatic disassociation. In an embodiment, the disassociating step comprises mechanical disassociation.

In an embodiment, the purifying step comprises removal of myelin from the brain cell composition. In an embodiment, the purifying step comprises removal of debris from the brain cell composition. In an embodiment, the purifying step comprises removal of red blood cells from the brain cell composition. In an embodiment, the purifying step comprises separation of the neural cells from glial cells. In an embodiment, the purifying step comprises use of papain.

In an embodiment, the culturing step comprises culturing the neural cells with laminin. In an embodiment, the culturing step comprises culturing the neural cells with Poly-D-Lysine (PDL).

In an embodiment, the neural cells comprise primary cells. In an embodiment, the neural cells comprise adult animal cells. In an embodiment, the adult animal cells are young adult animal cells. In an embodiment, the adult animal cells are middle-aged adult animal cells. In an embodiment, the adult animal cells are senior adult animal cells.

The age of the young adult animal cells, the middle-aged adult animal cells, and the senior adult animal cells can vary depending on the species of the animal. For instance, Table 1 includes various species and the ages of young adult animal cells, middle-aged adult animal cells, and senior adult animal cells in years:

TABLE 1

| | Young Adult | Middle-Aged | Senior Adult |
|---|---|---|---|
| | Cells | Adult | Cells |
| Species | Age (Years) | Cells Age (Years) | Age (Years) |
| mouse | 0-0.2 | 0.2-1.5 | 1.5-3 |
| rat | 0-0.2 | 0.2-1.5 | 1.5-3 |
| horse | 0-6 | 6-20 | 20-30 |
| pig | 0-1 | 1-11 | 11-22 |
| cow | 0-2 | 2-12 | 12-22 |
| goat | 0-1 | 1-8 | 8-15 |
| sheep | 0-1 | 1-8 | 8-15 |

In an embodiment, the adult animal cells are from an age of between 0-0.2 years. In an embodiment, the adult animal cells are from an age of between 0-1 years. In an embodiment, the adult animal cells are from an age of between 0-2 years. In an embodiment, the adult animal cells are from an age of between 0-6 years. In an embodiment, the adult animal cells are from an age of between 0.2-1.5 years. In an embodiment, the adult animal cells are from an age of between 1.5-3 years. In an embodiment, the adult animal cells are from an age of between 1-8 years. In an embodiment, the adult animal cells are from an age of between 1-11 years. In an embodiment, the adult animal cells are from an age of between 2-12 years. In an embodiment, the adult animal cells are from an age of between 6-20 years. In an embodiment, the adult animal cells are from an age of between 8-15 years. In an embodiment, the adult animal cells are from an age of between 11-22 years. In an embodiment, the adult animal cells are from an age of between 12-22 years. In an embodiment, the adult animal cells are from an age of between 20-30 years.

In an embodiment, the neural cells comprise large animal cells. In an embodiment, the neural cells are derived from a mammal. In an embodiment, the neural cells are derived from a livestock mammal. In an embodiment, the neural cells are derived from a sheep. In an embodiment, the neural cells are derived from a cow. In an embodiment, the neural cells are derived from a horse. In an embodiment, the neural cells are derived from a pig. In an embodiment, the neural cells are derived from a goat. In an embodiment, the neural cells are derived from a primate. In an embodiment, the neural cells are derived from a monkey.

In an embodiment, the neural cells are derived from a male animal. In an embodiment, the neural cells are derived from a female animal.

In an embodiment, the neural cells are central nervous system-derived cells. In an embodiment, the nervous system-derived cells comprise cells from one or more brain regions. In an embodiment, the nervous system-derived cells comprise cells from a spinal region. In an embodiment, the neural cells comprise cortical cells. In an embodiment, the neural cells comprise cortical astrocyte cells. In an embodiment, the neural cells comprise cortical neuron cells. In an embodiment, the neural cells comprise spinal cells. In an embodiment, the neural cells comprise spinal astrocyte cells. In an embodiment, the neural cells comprise spinal neuron cells. In an embodiment, the neural cells comprise hippocampal cells. In an embodiment, the neural cells comprise hippocampal astrocyte cells. In an embodiment, the neural cells comprise hippocampal neuron cells.

In an embodiment, the method is capable of processing over 1 million neural cells. In an embodiment, the method is capable of processing over 5 million neural cells. In an embodiment, the method is capable of processing over 10 million neural cells. In an embodiment, the method is capable of processing over 50 million neural cells. In an embodiment, the method is capable of processing over 100 million neural cells. In an embodiment, the method is capable of processing over 250 million neural cells. In an embodiment, the method is capable of processing over 500 million neural cells. In an embodiment, the method is capable of processing over 750 million neural cells. In an embodiment, the method is capable of processing over 1 billion neural cells.

In an illustrative aspect, a method of processing rodent neural cells is provided. The method comprises the steps of extracting one or more brain components from a rodent, dissociating cells from the brain components to form a brain cell composition, purifying the neural cells from the brain cell composition, performing magnetic cell separation (MACS) on the neural cells, and culturing the neural cells.

The step of magnetic cell separation (MACS) is well known to the skilled artisan. In an embodiment, the disassociating step comprises enzymatic disassociation. In an embodiment, the disassociating step comprises mechanical disassociation.

In an embodiment, the purifying step comprises removal of myelin from the brain cell composition. In an embodiment, the purifying step comprises removal of debris from the brain cell composition. In an embodiment, the purifying step comprises removal of red blood cells from the brain cell composition. In an embodiment, the purifying step comprises separation of the neural cells from glial cells. In an embodiment, the purifying step comprises use of papain.

In an embodiment, the culturing step comprises culturing the neural cells with laminin. In an embodiment, the culturing step comprises culturing the neural cells with Poly-D-Lysine (PDL).

In an embodiment, the neural cells comprise primary cells. In an embodiment, the neural cells comprise adult animal cells. In an embodiment, the adult animal cells are young adult animal cells. In an embodiment, the adult animal cells are middle-aged adult animal cells. In an embodiment, the adult animal cells are senior adult animal cells.

In an embodiment, the adult animal cells are from an age of between 0-0.2 years. In an embodiment, the adult animal cells are from an age of between 0-1 years. In an embodiment, the adult animal cells are from an age of between 0-2 years. In an embodiment, the adult animal cells are from an age of between 0-6 years. In an embodiment, the adult animal cells are from an age of between 0.2-1.5 years. In an embodiment, the adult animal cells are from an age of between 1.5-3 years. In an embodiment, the adult animal cells are from an age of between 1-8 years. In an embodiment, the adult animal cells are from an age of between 1-11 years. In an embodiment, the adult animal cells are from an age of between 2-12 years. In an embodiment, the adult animal cells are from an age of between 6-20 years. In an embodiment, the adult animal cells are from an age of between 8-15 years.

In an embodiment, the rodent is a mouse. In an embodiment, the rodent is a rat. In an embodiment, the rodent is a mouse or a rat of any genotype.

In an embodiment, the neural cells comprise large animal cells. In an embodiment, the neural cells are derived from a mammal. In an embodiment, the neural cells are derived from a livestock mammal. In an embodiment, the neural cells are derived from a sheep. In an embodiment, the neural cells are derived from a cow. In an embodiment, the neural cells are derived from a horse. In an embodiment, the neural cells are derived from a pig. In an embodiment, the neural cells are derived from a goat. In an embodiment, the neural cells are derived from a primate. In an embodiment, the neural cells are derived from a monkey.

In an embodiment, the neural cells are derived from a male animal. In an embodiment, the neural cells are derived from a female animal.

In an embodiment, the neural cells are central nervous system-derived cells. In an embodiment, the nervous system-derived cells comprise cells from one or more brain regions. In an embodiment, the nervous system-derived cells comprise cells from a spinal region. In an embodiment, the neural cells comprise cortical cells. In an embodiment, the neural cells comprise cortical astrocyte cells. In an embodiment, the neural cells comprise cortical neuron cells. In an embodiment, the neural cells comprise spinal cells. In an embodiment, the neural cells comprise spinal astrocyte cells. In an embodiment, the neural cells comprise spinal neuron cells. In an embodiment, the neural cells comprise hippocampal cells. In an embodiment, the neural cells comprise hippocampal astrocyte cells. In an embodiment, the neural cells comprise hippocampal neuron cells.

In an embodiment, the method is capable of processing over 1 million neural cells. In an embodiment, the method is capable of processing over 5 million neural cells. In an embodiment, the method is capable of processing over 10 million neural cells. In an embodiment, the method is capable of processing over 50 million neural cells. In an embodiment, the method is capable of processing over 100 million neural cells. In an embodiment, the method is capable of processing over 250 million neural cells. In an embodiment, the method is capable of processing over 500 million neural cells. In an embodiment, the method is capable of processing over 750 million neural cells. In an embodiment, the method is capable of processing over 1 billion neural cells.

In an illustrative aspect, an in vitro assay is provided. The in vitro assay comprises neural cells and means for performing the in vitro assay on using the neural cells. In an embodiment, the neural cells are obtained from the any one of the methods of processing neural cells as described herein. In an embodiment, the neural cells are obtained from the any one of the methods of processing rodent neural cells as described herein.

In an embodiment, the in vitro assay is a high-throughput screening assay. In an embodiment, the in vitro assay is a compound screening assay. In an embodiment, the in vitro assay is a therapeutic screening assay. For instance, the in vitro assays can utilize the neural cells of the present disclosure to replace embryonic or other “younger” cells currently utilized in compound or therapeutic screening assays. The adult neural cells according to the present disclosure can be advantageous when utilized in this manner.

In an embodiment, the therapeutic screening assay identifies a drug candidate for a neurological disease or disorder. In an embodiment, the therapeutic screening assay identifies a drug candidate for a neurodegenerative disease. In an embodiment, the therapeutic screening assay identifies a drug candidate for a neurotraumatic injury. In an embodiment, the therapeutic screening assay identifies a drug candidate for an age-associated disorder. In an embodiment, the therapeutic screening assay identifies a drug candidate for normal aging. In an embodiment, the therapeutic screening assay identifies a drug candidate for a disease that decreases neuron survival. In an embodiment, the therapeutic screening assay identifies a drug candidate for a disease that decreases neurite outgrowth. In an embodiment, the therapeutic screening assay identifies a drug candidate for a disease that decreases synaptic plasticity. In an embodiment, the therapeutic screening assay identifies a drug candidate for Alzheimer's Disease. In an embodiment, the therapeutic screening assay identifies a drug candidate for a spinal cord-related disease. In an embodiment, the therapeutic screening assay identifies a drug candidate for spinal cord injury. In an embodiment, the therapeutic screening assay identifies a drug candidate for spinal cord infection. In an embodiment, the therapeutic screening assay identifies a drug candidate for spinal cord degradation. In an embodiment, the therapeutic screening assay identifies a drug candidate for traumatic brain injury. In an embodiment, the therapeutic screening assay identifies a drug candidate for paralysis. In an embodiment, the therapeutic screening assay identifies a drug candidate for motor function. In an embodiment, the therapeutic screening assay identifies a drug candidate for glaucoma. In an embodiment, the therapeutic screening assay identifies a drug candidate for Parkinson's Disease. In an embodiment, the therapeutic screening assay identifies a drug candidate for stroke.

In an embodiment, the in vitro assay is a metabolic assay. In an embodiment, the in vitro assay is a siRNA-based assay. In an embodiment, the in vitro assay is a gene targeting assay. In an embodiment, the in vitro assay is a transfection efficiency assay. In an embodiment, the in vitro assay is a scratch assay. In an embodiment, the in vitro assay is a cell migration assay. In an embodiment, the in vitro assay is a cell morphology assay. In an embodiment, the in vitro assay is a wound formation assay.

In an embodiment, the in vitro assay is an RNA-based assay. In an embodiment, the RNA-based assay is an assay utilizing RNA selected from the group consisting of mRNA, pre-mRNA, tRNA, rRNA, snRNA, aRNA, siRNA, miRNA, RNAi, and tmRNA.

In an embodiment, the in vitro assay is a survival assay. In an embodiment, the in vitro assay is a toxicity assay. In an embodiment, the in vitro assay is a regeneration assay. In an embodiment, the in vitro assay evaluates neural cell regeneration. In an embodiment, the in vitro assay evaluates neural cell survival. In an embodiment, the in vitro assay evaluates neural cell growth.

In an illustrative aspect, a kit is provided. The kit comprises neural cells and means for performing an in vitro assay using the neural cells. In an embodiment, the neural cells are obtained from the any one of the methods of processing neural cells as described herein. In an embodiment, the neural cells are obtained from the any one of the methods of processing rodent neural cells as described herein.

In an embodiment, the in vitro assay is a high-throughput screening assay. In an embodiment, the in vitro assay is a compound screening assay. In an embodiment, the in vitro assay is a therapeutic screening assay. For instance, the in vitro assays can utilize the neural cells of the present disclosure to replace embryonic or other “younger” cells currently utilized in compound or therapeutic screening assays. The adult neural cells according to the present disclosure can be advantageous when utilized in this manner.

In an embodiment, the therapeutic screening assay identifies a drug candidate for a neurological disease or disorder. In an embodiment, the therapeutic screening assay identifies a drug candidate for a neurodegenerative disease. In an embodiment, the therapeutic screening assay identifies a drug candidate for a neurotraumatic injury. In an embodiment, the therapeutic screening assay identifies a drug candidate for an age-associated disorder. In an embodiment, the therapeutic screening assay identifies a drug candidate for normal aging. In an embodiment, the therapeutic screening assay identifies a drug candidate for a disease that decreases neuron survival. In an embodiment, the therapeutic screening assay identifies a drug candidate for a disease that decreases neurite outgrowth. In an embodiment, the therapeutic screening assay identifies a drug candidate for a disease that decreases synaptic plasticity. In an embodiment, the therapeutic screening assay identifies a drug candidate for Alzheimer's Disease. In an embodiment, the therapeutic screening assay identifies a drug candidate for a spinal cord-related disease. In an embodiment, the therapeutic screening assay identifies a drug candidate for spinal cord injury. In an embodiment, the therapeutic screening assay identifies a drug candidate for spinal cord infection. In an embodiment, the therapeutic screening assay identifies a drug candidate for spinal cord degradation. In an embodiment, the therapeutic screening assay identifies a drug candidate for traumatic brain injury. In an embodiment, the therapeutic screening assay identifies a drug candidate for paralysis. In an embodiment, the therapeutic screening assay identifies a drug candidate for motor function. In an embodiment, the therapeutic screening assay identifies a drug candidate for glaucoma. In an embodiment, the therapeutic screening assay identifies a drug candidate for Parkinson's Disease. In an embodiment, the therapeutic screening assay identifies a drug candidate for stroke.

In an embodiment, the in vitro assay is a metabolic assay. In an embodiment, the in vitro assay is a siRNA-based assay. In an embodiment, the in vitro assay is a gene targeting assay. In an embodiment, the in vitro assay is a transfection efficiency assay. In an embodiment, the in vitro assay is a scratch assay. In an embodiment, the in vitro assay is a cell migration assay. In an embodiment, the in vitro assay is a cell morphology assay. In an embodiment, the in vitro assay is a wound formation assay.

In an embodiment, the in vitro assay is an RNA-based assay. In an embodiment, the RNA-based assay is an assay utilizing RNA selected from the group consisting of mRNA, pre-mRNA, tRNA, rRNA, snRNA, aRNA, siRNA, miRNA, RNAi, and tmRNA.

In an embodiment, the in vitro assay is a survival assay. In an embodiment, the in vitro assay is a toxicity assay. In an embodiment, the in vitro assay is a regeneration assay. In an embodiment, the in vitro assay evaluates neural cell regeneration. In an embodiment, the in vitro assay evaluates neural cell survival. In an embodiment, the in vitro assay evaluates neural cell growth.

The following numbered embodiments are contemplated and are non-limiting:

1. A method of processing neural cells, said method comprising the steps of:
extracting one or more brain components from an animal,
dissociating cells from the brain components to form a brain cell composition,
purifying the neural cells from the brain cell composition, and
culturing the neural cells.

2. The method of clause 1, any other suitable clause, or any combination of suitable clauses, wherein the disassociating step comprises enzymatic disassociation.

3. The method of clause 1, any other suitable clause, or any combination of suitable clauses, wherein the disassociating step comprises mechanical disassociation.

4. The method of clause 1, any other suitable clause, or any combination of suitable clauses, wherein the purifying step comprises removal of myelin from the brain cell composition.

5. The method of clause 1, any other suitable clause, or any combination of suitable clauses, wherein the purifying step comprises removal of debris from the brain cell composition.

6. The method of clause 1, any other suitable clause, or any combination of suitable clauses, wherein the purifying step comprises removal of red blood cells from the brain cell composition.

7. The method of clause 1, any other suitable clause, or any combination of suitable clauses, wherein the purifying step comprises separation of the neural cells from glial cells.

8. The method of clause 1, any other suitable clause, or any combination of suitable clauses, wherein the purifying step comprises use of papain.

9. The method of clause 1, any other suitable clause, or any combination of suitable clauses, wherein the culturing step comprises culturing the neural cells with laminin.

10. The method of clause 1, any other suitable clause, or any combination of suitable clauses, wherein the culturing step comprises culturing the neural cells with Poly-D-Lysine (PDL).

11. The method of clause 1, any other suitable clause, or any combination of suitable clauses, wherein the neural cells comprise primary cells.

12. The method of clause 1, any other suitable clause, or any combination of suitable clauses, wherein the neural cells comprise adult animal cells.

13. The method of clause 12, any other suitable clause, or any combination of suitable clauses, wherein the adult animal cells are young adult animal cells.

14. The method of clause 12, any other suitable clause, or any combination of suitable clauses, wherein the adult animal cells are middle-aged adult animal cells.

15. The method of clause 12, any other suitable clause, or any combination of suitable clauses, wherein the adult animal cells are senior adult animal cells.

16. The method of clause 12, any other suitable clause, or any combination of suitable clauses, wherein the adult animal cells are from an age of between 0-0.2 years.

17. The method of clause 12, any other suitable clause, or any combination of suitable clauses, wherein the adult animal cells are from an age of between 0-1 years.

18. The method of clause 12, any other suitable clause, or any combination of suitable clauses, wherein the adult animal cells are from an age of between 0-2 years.

19. The method of clause 12, any other suitable clause, or any combination of suitable clauses, wherein the adult animal cells are from an age of between 0-6 years.

20. The method of clause 12, any other suitable clause, or any combination of suitable clauses, wherein the adult animal cells are from an age of between 0.2-1.5 years.

21. The method of clause 12, any other suitable clause, or any combination of suitable clauses, wherein the adult animal cells are from an age of between 1.5-3 years.

22. The method of clause 12, any other suitable clause, or any combination of suitable clauses, wherein the adult animal cells are from an age of between 1-8 years.

23. The method of clause 12, any other suitable clause, or any combination of suitable clauses, wherein the adult animal cells are from an age of between 1-11 years.

24. The method of clause 12, any other suitable clause, or any combination of suitable clauses, wherein the adult animal cells are from an age of between 2-12 years.

25. The method of clause 12, any other suitable clause, or any combination of suitable clauses, wherein the adult animal cells are from an age of between 6-20 years.

26. The method of clause 12, any other suitable clause, or any combination of suitable clauses, wherein the adult animal cells are from an age of between 8-15 years.

27. The method of clause 12, any other suitable clause, or any combination of suitable clauses, wherein the adult animal cells are from an age of between 11-22 years.

28. The method of clause 12, any other suitable clause, or any combination of suitable clauses, wherein the adult animal cells are from an age of between 12-22 years.

29. The method of clause 12, any other suitable clause, or any combination of suitable clauses, wherein the adult animal cells are from an age of between 20-30 years.

30. The method of clause 1, any other suitable clause, or any combination of suitable clauses, wherein the neural cells comprise large animal cells.

31. The method of clause 1, any other suitable clause, or any combination of suitable clauses, wherein the neural cells are derived from a mammal.

32. The method of clause 1, any other suitable clause, or any combination of suitable clauses, wherein the neural cells are derived from a livestock mammal.

33. The method of clause 1, any other suitable clause, or any combination of suitable clauses, wherein the neural cells are derived from a sheep.

34. The method of clause 1, any other suitable clause, or any combination of suitable clauses, wherein the neural cells are derived from a cow.

35. The method of clause 1, any other suitable clause, or any combination of suitable clauses, wherein the neural cells are derived from a horse.

36. The method of clause 1, any other suitable clause, or any combination of suitable clauses, wherein the neural cells are derived from a pig.

37. The method of clause 1, any other suitable clause, or any combination of suitable clauses, wherein the neural cells are derived from a goat.

38. The method of clause 1, any other suitable clause, or any combination of suitable clauses, wherein the neural cells are derived from a primate.

39. The method of clause 1, any other suitable clause, or any combination of suitable clauses, wherein the neural cells are derived from a monkey.

40. The method of clause 1, any other suitable clause, or any combination of suitable clauses, wherein the neural cells are derived from a male animal.

41. The method of clause 1, any other suitable clause, or any combination of suitable clauses, wherein the neural cells are derived from a female animal.

42. The method of clause 1, any other suitable clause, or any combination of suitable clauses, wherein the neural cells are central nervous system-derived cells.

43. The method of clause 42, any other suitable clause, or any combination of suitable clauses, wherein the nervous system-derived cells comprise cells from one or more brain regions.

44. The method of clause 42, any other suitable clause, or any combination of suitable clauses, wherein the nervous system-derived cells comprise cells from a spinal region.

45. The method of clause 1, any other suitable clause, or any combination of suitable clauses, wherein the neural cells comprise cortical cells.

46. The method of clause 1, any other suitable clause, or any combination of suitable clauses, wherein the neural cells comprise cortical astrocyte cells.

47. The method of clause 1, any other suitable clause, or any combination of suitable clauses, wherein the neural cells comprise cortical neuron cells.

48. The method of clause 1, any other suitable clause, or any combination of suitable clauses, wherein the neural cells comprise spinal cells.

49. The method of clause 1, any other suitable clause, or any combination of suitable clauses, wherein the neural cells comprise spinal astrocyte cells.

50. The method of clause 1, any other suitable clause, or any combination of suitable clauses, wherein the neural cells comprise spinal neuron cells.

51. The method of clause 1, any other suitable clause, or any combination of suitable clauses, wherein the neural cells comprise hippocampal cells.

52. The method of clause 1, any other suitable clause, or any combination of suitable clauses, wherein the neural cells comprise hippocampal astrocyte cells.

53. The method of clause 1, any other suitable clause, or any combination of suitable clauses, wherein the neural cells comprise hippocampal neuron cells.

54. The method of clause 1, any other suitable clause, or any combination of suitable clauses, wherein the method is capable of processing over 1 million neural cells.

55. The method of clause 1, any other suitable clause, or any combination of suitable clauses, wherein the method is capable of processing over 5 million neural cells.

56. The method of clause 1, any other suitable clause, or any combination of suitable clauses, wherein the method is capable of processing over 10 million neural cells.

57. The method of clause 1, any other suitable clause, or any combination of suitable clauses, wherein the method is capable of processing over 50 million neural cells.

58. The method of clause 1, any other suitable clause, or any combination of suitable clauses, wherein the method is capable of processing over 100 million neural cells.

59. The method of clause 1, any other suitable clause, or any combination of suitable clauses, wherein the method is capable of processing over 250 million neural cells.

60. The method of clause 1, any other suitable clause, or any combination of suitable clauses, wherein the method is capable of processing over 500 million neural cells.

61. The method of clause 1, any other suitable clause, or any combination of suitable clauses, wherein the method is capable of processing over 750 million neural cells.

62. The method of clause 1, any other suitable clause, or any combination of suitable clauses, wherein the method is capable of processing over 1 billion neural cells.

63. A method of processing rodent neural cells, said method comprising the steps of:
extracting one or more brain components from a rodent,
dissociating cells from the brain components to form a brain cell composition,
purifying the neural cells from the brain cell composition,
performing magnetic cell separation (MACS) on the neural cells, and
culturing the neural cells.

64. The method of clause 63, any other suitable clause, or any combination of suitable clauses, wherein the disassociating step comprises enzymatic disassociation.

65. The method of clause 63, any other suitable clause, or any combination of suitable clauses, wherein the disassociating step comprises mechanical disassociation.

66. The method of clause 63, any other suitable clause, or any combination of suitable clauses, wherein the purifying step comprises removal of myelin from the brain cell composition.

67. The method of clause 63, any other suitable clause, or any combination of suitable clauses, wherein the purifying step comprises removal of debris from the brain cell composition.

68. The method of clause 63, any other suitable clause, or any combination of suitable clauses, wherein the purifying step comprises removal of red blood cells from the brain cell composition.

69. The method of clause 63, any other suitable clause, or any combination of suitable clauses, wherein the purifying step comprises separation of the neural cells from glial cells.

70. The method of clause 63, any other suitable clause, or any combination of suitable clauses, wherein the purifying step comprises use of papain.

71. The method of clause 63, any other suitable clause, or any combination of suitable clauses, wherein the culturing step comprises culturing the neural cells with laminin.

72. The method of clause 63, any other suitable clause, or any combination of suitable clauses, wherein the culturing step comprises culturing the neural cells with Poly-D-Lysine (PDL).

73. The method of clause 63, any other suitable clause, or any combination of suitable clauses, wherein the neural cells comprise primary cells.

74. The method of clause 63, any other suitable clause, or any combination of suitable clauses, wherein the neural cells comprise adult animal cells.

75. The method of clause 74, any other suitable clause, or any combination of suitable clauses, wherein the adult animal cells are young adult animal cells.

76. The method of clause 74, any other suitable clause, or any combination of suitable clauses, wherein the adult animal cells are middle-aged adult animal cells.

77. The method of clause 74, any other suitable clause, or any combination of suitable clauses, wherein the adult animal cells are senior adult animal cells.

78. The method of clause 74, any other suitable clause, or any combination of suitable clauses, wherein the adult animal cells are from an age of between 0-0.2 years.

79. The method of clause 74, any other suitable clause, or any combination of suitable clauses, wherein the adult animal cells are from an age of between 0-1 years.

80. The method of clause 74, any other suitable clause, or any combination of suitable clauses, wherein the adult animal cells are from an age of between 0-2 years.

81. The method of clause 74, any other suitable clause, or any combination of suitable clauses, wherein the adult animal cells are from an age of between 0-6 years.

82. The method of clause 74, any other suitable clause, or any combination of suitable clauses, wherein the adult animal cells are from an age of between 0.2-1.5 years.

83. The method of clause 74, any other suitable clause, or any combination of suitable clauses, wherein the adult animal cells are from an age of between 1.5-3 years.

84. The method of clause 74, any other suitable clause, or any combination of suitable clauses, wherein the adult animal cells are from an age of between 1-8 years.

85. The method of clause 74, any other suitable clause, or any combination of suitable clauses, wherein the adult animal cells are from an age of between 1-11 years.

86. The method of clause 74, any other suitable clause, or any combination of suitable clauses, wherein the adult animal cells are from an age of between 2-12 years.

87. The method of clause 74, any other suitable clause, or any combination of suitable clauses, wherein the adult animal cells are from an age of between 6-20 years.

88. The method of clause 74, any other suitable clause, or any combination of suitable clauses, wherein the adult animal cells are from an age of between 8-15 years.

89. The method of clause 63, any other suitable clause, or any combination of suitable clauses, wherein the rodent is a mouse.

90. The method of clause 63, any other suitable clause, or any combination of suitable clauses, wherein the rodent is a rat.

91. The method of clause 63, any other suitable clause, or any combination of suitable clauses, wherein the rodent is a mouse or a rat of any genotype.

92. The method of clause 63, any other suitable clause, or any combination of suitable clauses, wherein the neural cells are derived from a male animal.

93. The method of clause 63, any other suitable clause, or any combination of suitable clauses, wherein the neural cells are derived from a female animal.

94. The method of clause 63, any other suitable clause, or any combination of suitable clauses, wherein the neural cells are central nervous system-derived cells.

95. The method of clause 94, any other suitable clause, or any combination of suitable clauses, wherein the nervous system-derived cells comprise cells from one or more brain regions.

96. The method of clause 94, any other suitable clause, or any combination of suitable clauses, wherein the nervous system-derived cells comprise cells from a spinal region.

97. The method of clause 63, any other suitable clause, or any combination of suitable clauses, wherein the neural cells comprise cortical cells.

98. The method of clause 63, any other suitable clause, or any combination of suitable clauses, wherein the neural cells comprise cortical astrocyte cells.

99. The method of clause 63, any other suitable clause, or any combination of suitable clauses, wherein the neural cells comprise cortical neuron cells.

100. The method of clause 63, any other suitable clause, or any combination of suitable clauses, wherein the neural cells comprise spinal cells.

101. The method of clause 63, any other suitable clause, or any combination of suitable clauses, wherein the neural cells comprise spinal astrocyte cells.

102. The method of clause 63, any other suitable clause, or any combination of suitable clauses, wherein the neural cells comprise spinal neuron cells.

103. The method of clause 63, any other suitable clause, or any combination of suitable clauses, wherein the neural cells comprise hippocampal cells.

104. The method of clause 63, any other suitable clause, or any combination of suitable clauses, wherein the neural cells comprise hippocampal astrocyte cells.

105. The method of clause 63, any other suitable clause, or any combination of suitable clauses, wherein the neural cells comprise hippocampal neuron cells.

106. The method of clause 63, any other suitable clause, or any combination of suitable clauses, wherein the method is capable of processing over 1 million neural cells.

107. The method of clause 63, any other suitable clause, or any combination of suitable clauses, wherein the method is capable of processing over 5 million neural cells.

108. The method of clause 63, any other suitable clause, or any combination of suitable clauses, wherein the method is capable of processing over 10 million neural cells.

109. The method of clause 63, any other suitable clause, or any combination of suitable clauses, wherein the method is capable of processing over 50 million neural cells.

110. The method of clause 63, any other suitable clause, or any combination of suitable clauses, wherein the method is capable of processing over 100 million neural cells.

111. The method of clause 63, any other suitable clause, or any combination of suitable clauses, wherein the method is capable of processing over 250 million neural cells.

112. The method of clause 63, any other suitable clause, or any combination of suitable clauses, wherein the method is capable of processing over 500 million neural cells.

113. The method of clause 63, any other suitable clause, or any combination of suitable clauses, wherein the method is capable of processing over 750 million neural cells.

114. The method of clause 63, any other suitable clause, or any combination of suitable clauses, wherein the method is capable of processing over 1 billion neural cells.

115. An in vitro assay comprising neural cells and means for performing the in vitro assay on using the neural cells.

116. The in vitro assay of clause 115, any other suitable clause, or any combination of suitable clauses, wherein the neural cells are obtained from the method of any one of clauses 1 to 62.

117. The in vitro assay of clause 115, any other suitable clause, or any combination of suitable clauses, wherein the neural cells are obtained from the method of any one of clauses 63 to 114.

118. The in vitro assay of clause 115, any other suitable clause, or any combination of suitable clauses, wherein the in vitro assay is a high-throughput screening assay.

119. The in vitro assay of clause 115, any other suitable clause, or any combination of suitable clauses, wherein the in vitro assay is a compound screening assay.

120. The in vitro assay of clause 115, any other suitable clause, or any combination of suitable clauses, wherein the in vitro assay is a therapeutic screening assay.

121. The in vitro assay of clause 120, any other suitable clause, or any combination of suitable clauses, wherein the therapeutic screening assay identifies a drug candidate for a neurological disease or disorder.

122. The in vitro assay of clause 120, any other suitable clause, or any combination of suitable clauses, wherein the therapeutic screening assay identifies a drug candidate for a neurodegenerative disease.

123. The in vitro assay of clause 120, any other suitable clause, or any combination of suitable clauses, wherein the therapeutic screening assay identifies a drug candidate for a neurotraumatic injury.

124. The in vitro assay of clause 120, any other suitable clause, or any combination of suitable clauses, wherein the therapeutic screening assay identifies a drug candidate for an age-associated disorder.

125. The in vitro assay of clause 120, any other suitable clause, or any combination of suitable clauses, wherein the therapeutic screening assay identifies a drug candidate for normal aging.

126. The in vitro assay of clause 120, any other suitable clause, or any combination of suitable clauses, wherein the therapeutic screening assay identifies a drug candidate for a disease that decreases neuron survival.

127. The in vitro assay of clause 120, any other suitable clause, or any combination of suitable clauses, wherein the therapeutic screening assay identifies a drug candidate for a disease that decreases neurite outgrowth.

128. The in vitro assay of clause 120, any other suitable clause, or any combination of suitable clauses, wherein the therapeutic screening assay identifies a drug candidate for a disease that decreases synaptic plasticity.

129. The in vitro assay of clause 120, any other suitable clause, or any combination of suitable clauses, wherein the therapeutic screening assay identifies a drug candidate for Alzheimer's Disease.

130. The in vitro assay of clause 120, any other suitable clause, or any combination of suitable clauses, wherein the therapeutic screening assay identifies a drug candidate for a spinal cord-related disease.

131. The in vitro assay of clause 120, any other suitable clause, or any combination of suitable clauses, wherein the therapeutic screening assay identifies a drug candidate for spinal cord injury.

132. The in vitro assay of clause 120, any other suitable clause, or any combination of suitable clauses, wherein the therapeutic screening assay identifies a drug candidate for spinal cord infection.

133. The in vitro assay of clause 120, any other suitable clause, or any combination of suitable clauses, wherein the therapeutic screening assay identifies a drug candidate for spinal cord degradation.

134. The in vitro assay of clause 120, any other suitable clause, or any combination of suitable clauses, wherein the therapeutic screening assay identifies a drug candidate for traumatic brain injury.

135. The in vitro assay of clause 120, any other suitable clause, or any combination of suitable clauses, wherein the therapeutic screening assay identifies a drug candidate for paralysis.

136. The in vitro assay of clause 120, any other suitable clause, or any combination of suitable clauses, wherein the therapeutic screening assay identifies a drug candidate for motor function.

137. The in vitro assay of clause 120, any other suitable clause, or any combination of suitable clauses, wherein the therapeutic screening assay identifies a drug candidate for glaucoma.

138. The in vitro assay of clause 120, any other suitable clause, or any combination of suitable clauses, wherein the therapeutic screening assay identifies a drug candidate for Parkinson's Disease.

139. The in vitro assay of clause 120, any other suitable clause, or any combination of suitable clauses, wherein the therapeutic screening assay identifies a drug candidate for stroke.

140. The in vitro assay of clause 115, any other suitable clause, or any combination of suitable clauses, wherein the in vitro assay is a metabolic assay.

141. The in vitro assay of clause 115, any other suitable clause, or any combination of suitable clauses, wherein the in vitro assay is a siRNA-based assay.

142. The in vitro assay of clause 115, any other suitable clause, or any combination of suitable clauses, wherein the in vitro assay is a gene targeting assay.

143. The in vitro assay of clause 115, any other suitable clause, or any combination of suitable clauses, wherein the in vitro assay is a transfection efficiency assay.

144. The in vitro assay of clause 115, any other suitable clause, or any combination of suitable clauses, wherein the in vitro assay is a scratch assay.

145. The in vitro assay of clause 115, any other suitable clause, or any combination of suitable clauses, wherein the in vitro assay is a cell migration assay.

146. The in vitro assay of clause 115, any other suitable clause, or any combination of suitable clauses, wherein the in vitro assay is a cell morphology assay.

147. The in vitro assay of clause 115, any other suitable clause, or any combination of suitable clauses, wherein the in vitro assay is a wound formation assay.

148. The in vitro assay of clause 115, any other suitable clause, or any combination of suitable clauses, wherein the in vitro assay is an RNA-based assay.

149. The in vitro assay of clause 148, any other suitable clause, or any combination of suitable clauses, wherein the RNA-based assay is an assay utilizing RNA selected from the group consisting of mRNA, pre-mRNA, tRNA, rRNA, snRNA, aRNA, siRNA, miRNA, RNAi, and tmRNA.

150. The in vitro assay of clause 115, any other suitable clause, or any combination of suitable clauses, wherein the in vitro assay is a survival assay.

151. The in vitro assay of clause 115, any other suitable clause, or any combination of suitable clauses, wherein the in vitro assay is a toxicity assay.

152. The in vitro assay of clause 115, any other suitable clause, or any combination of suitable clauses, wherein the in vitro assay is a regeneration assay.

153. The in vitro assay of clause 115, any other suitable clause, or any combination of suitable clauses, wherein the in vitro assay evaluates neural cell regeneration.

154. The in vitro assay of clause 115, any other suitable clause, or any combination of suitable clauses, wherein the in vitro assay evaluates neural cell survival.

155. The in vitro assay of clause 115, any other suitable clause, or any combination of suitable clauses, wherein the in vitro assay evaluates neural cell growth.

156. A kit comprising neural cells and means for performing an in vitro assay using the neural cells.

157. The kit of clause 156, any other suitable clause, or any combination of suitable clauses, wherein the neural cells are obtained from the method of any one of clauses 1 to 62.

158. The kit of clause 156, any other suitable clause, or any combination of suitable clauses, wherein the neural cells are obtained from the method of any one of clauses 63 to 114.

159. The kit of clause 156, any other suitable clause, or any combination of suitable clauses, wherein the in vitro assay is a high-throughput screening assay.

160. The kit of clause 156, any other suitable clause, or any combination of suitable clauses, wherein the in vitro assay is a compound screening assay.

161. The kit of clause 156, any other suitable clause, or any combination of suitable clauses, wherein the in vitro assay is a therapeutic screening assay.

162. The kit of clause 161, any other suitable clause, or any combination of suitable clauses, wherein the therapeutic screening assay identifies a drug candidate for a neurological disease or disorder

163. The kit of clause 161, any other suitable clause, or any combination of suitable clauses, wherein the therapeutic screening assay identifies a drug candidate for a neurodegenerative disease.

164. The kit of clause 161, any other suitable clause, or any combination of suitable clauses, wherein the therapeutic screening assay identifies a drug candidate for a neurotraumatic injury.

165. The kit of clause 161, any other suitable clause, or any combination of suitable clauses, wherein the therapeutic screening assay identifies a drug candidate for an age-associated disorder.

166. The kit of clause 161, any other suitable clause, or any combination of suitable clauses, wherein the therapeutic screening assay identifies a drug candidate for normal aging.

167. The kit of clause 161, any other suitable clause, or any combination of suitable clauses, wherein the therapeutic screening assay identifies a drug candidate for a disease that decreases neuron survival.

168. The kit of clause 161, any other suitable clause, or any combination of suitable clauses, wherein the therapeutic screening assay identifies a drug candidate for a disease that decreases neurite outgrowth.

169. The kit of clause 161, any other suitable clause, or any combination of suitable clauses, wherein the therapeutic screening assay identifies a drug candidate for a disease that decreases synaptic plasticity.

170. The kit of clause 161, any other suitable clause, or any combination of suitable clauses, wherein the therapeutic screening assay identifies a drug candidate for Alzheimer's Disease.

171. The kit of clause 161, any other suitable clause, or any combination of suitable clauses, wherein the therapeutic screening assay identifies a drug candidate for a spinal cord-related disease.

172. The kit of clause 161, any other suitable clause, or any combination of suitable clauses, wherein the therapeutic screening assay identifies a drug candidate for spinal cord injury.

173. The kit of clause 161, any other suitable clause, or any combination of suitable clauses, wherein the therapeutic screening assay identifies a drug candidate for spinal cord infection.

174. The kit of clause 161, any other suitable clause, or any combination of suitable clauses, wherein the therapeutic screening assay identifies a drug candidate for spinal cord degradation.

175. The kit of clause 161, any other suitable clause, or any combination of suitable clauses, wherein the therapeutic screening assay identifies a drug candidate for traumatic brain injury.

176. The kit of clause 161, any other suitable clause, or any combination of suitable clauses, wherein the therapeutic screening assay identifies a drug candidate for paralysis.

177. The kit of clause 161, any other suitable clause, or any combination of suitable clauses, wherein the therapeutic screening assay identifies a drug candidate for motor function.

178. The kit of clause 161, any other suitable clause, or any combination of suitable clauses, wherein the therapeutic screening assay identifies a drug candidate for glaucoma.

179. The kit of clause 161, any other suitable clause, or any combination of suitable clauses, wherein the therapeutic screening assay identifies a drug candidate for Parkinson's Disease.

180. The kit of clause 161, any other suitable clause, or any combination of suitable clauses, wherein the therapeutic screening assay identifies a drug candidate for stroke.

181. The kit of clause 156, any other suitable clause, or any combination of suitable clauses, wherein the in vitro assay is a metabolic assay.

182. The kit of clause 156, any other suitable clause, or any combination of suitable clauses, wherein the in vitro assay is a siRNA-based assay.

183. The kit of clause 156, any other suitable clause, or any combination of suitable clauses, wherein the in vitro assay is a gene targeting assay.

184. The kit of clause 156, any other suitable clause, or any combination of suitable clauses, wherein the in vitro assay is a transfection efficiency assay.

185. The kit of clause 156, any other suitable clause, or any combination of suitable clauses, wherein the in vitro assay is a scratch assay.

186. The kit of clause 156, any other suitable clause, or any combination of suitable clauses, wherein the in vitro assay is a cell migration assay.

187. The kit of clause 156, any other suitable clause, or any combination of suitable clauses, wherein the in vitro assay is a cell morphology assay.

188. The kit of clause 156, any other suitable clause, or any combination of suitable clauses, wherein the in vitro assay is a wound formation assay.

189. The kit of clause 156, any other suitable clause, or any combination of suitable clauses, wherein the in vitro assay is an RNA-based assay.

190. The kit of clause 189, any other suitable clause, or any combination of suitable clauses, wherein the RNA-based assay is an assay utilizing RNA selected from the group consisting of mRNA, pre-mRNA, tRNA, rRNA, snRNA, aRNA, siRNA, miRNA, RNAi, and tmRNA.

191. The kit of clause 156, any other suitable clause, or any combination of suitable clauses, wherein the in vitro assay is a survival assay.

192. The kit of clause 156, any other suitable clause, or any combination of suitable clauses, wherein the in vitro assay is a toxicity assay.

193. The kit of clause 156, any other suitable clause, or any combination of suitable clauses, wherein the in vitro assay is a regeneration assay.

194. The kit of clause 156, any other suitable clause, or any combination of suitable clauses, wherein the in vitro assay evaluates neural cell regeneration.

195. The kit of clause 156, any other suitable clause, or any combination of suitable clauses, wherein the in vitro assay evaluates neural cell survival.

196. The kit of clause 156, any other suitable clause, or any combination of suitable clauses, wherein the in vitro assay evaluates neural cell growth.

### Example 1

### Processing and Screening Method for Adult Mice Cortical Neurons

The instant example provides an exemplary protocol, method, and application of processing and screening using adult cortical neurons obtained from adult mice. In particular, the instant example used young adult and middle-aged male and female wild-type C57Bl/6 mice in which the young adult group comprised mice of 4-9 weeks of age and the middle-age group comprised mice of 40-48 weeks of age.

### Cell Culture:

Approximately 20 μL of 50 μg/mL Poly-D-Lysine (PDL, Sigma Aldrich, A-003-M) was added onto the wells of 384-well glass bottom (Brooks Life Sciences, MGB101-1-2-LG-L) or plastic bottom (Greiner-Bio, 781091) plates and incubated inside a 5% CO₂ incubator at 37° C. for 48 hours. After incubation, the wells of the plates were washed 5 times with H₂O and set to dry overnight at room temperature and were used within 24 hours after drying.

Mice were euthanized and their brains were extracted and placed in cold Hank's balanced salt solution (HBSS) followed by microdissection of the cortex. Up to 1.25 grams of cortical tissue were placed in gentleMACS™ C tubes (Miltenyi Biotec, #130-093-237), where each tube contained 5 mL of 0.3 mg/mL papain (Worthington, LS003126) diluted in HBSS.

The gentleMACS™ C tubes were placed on the gentleMACS™ Octo Dissociator with heaters (Miltenyi Biotec, #130-096-427) with heating cuffs attached and underwent the gentleMACS Program 37C_ABDK_01 protocol. After protocol completion, the contents of the gentleMACS™ C Tube were strained through a 70 μm cell strainer (Miltenyi Biotec, #130-110-916) placed on top of a 15 mL conical centrifuge tube. Then, 7 mL of cold Dulbecco's Phosphate-Buffered Saline with glucose and pyruvate (DPBS, Thermo Fisher Scientific, 14287072) was added into each of the 15 mL conical centrifuge tubes on top of the strained cells. The 15 mL tubes were centrifuged at 300×g for 10 minutes at 4° C. before aspirating the supernatant completely.

Debris removal solution was made by adding 1800 μL of Debris Removal Concentrate (Miltenyi Biotec, #130-109-398) to 6200 μL of cold DPBS. The remaining pellet inside the 15 mL tubes was resuspended with 8 mL of Debris Removal Solution. Very slowly, 4 mL of cold DPBS was dispensed on top of the debris removal solution and cell mixture in each 15 mL tube forming a clear layer on top. The 15 mL tubes were centrifuged at 3000×g for 10 minutes at 4° C. with slow acceleration and deceleration. The top clear and middle debris layers were aspirated leaving the milky mixture beneath the debris layer untouched. 6 mL of DPBS was added onto the milky mixture and mixed gently before centrifuging at 300×g for 5 minutes at 4° C. All supernatant was aspirated afterwards.

Red Blood Cell Remover Solution was made by mixing 125 μL of Red Blood Cell Lysis Solution 10× (Miltenyi Biotec, #130-094-183) with 1125 μL of H₂O. The remaining pellet was resuspended in 1.25 mL of Red Blood Cell Remover Solution and incubated for 10 minutes at 4° C. before the addition of 12 mL of 0.5% bovine serum albumin (BSA, Miltenyi Biotec, #130-091-376) diluted in DPBS. The mixture was centrifuged at 300×g for 5 minutes at 4° C. with the supernatant aspirated completely afterwards. The remaining pellet was resuspended in 80 μL of 0.5% BSA and 20 μL of Non-Neuronal Cells Biotin-Antibody Cocktail (Miltenyi Biotec, #130-115-389) and incubated for 5 minutes at 4° C. Cells were washed by adding 2 mL of 0.5% BSA followed by centrifugation at 300×g for 5 minutes at 4° C. followed by aspiration of the supernatant. The remaining pellet was resuspended in 80 μL of 0.5% BSA and 20 μL of Anti-Biotin MicroBeads (Miltenyi Biotec, #130-115-389) and incubated for 10 minutes at 4° C.

After the addition of 6 mL 0.5% BSA, the mixture was flowed through 0.5% BSA primed LS columns (Miltenyi Biotec, #130-042-401). The negative fraction containing the majority of neurons was collected and centrifuged at 300×g for 5 minutes at 4° C. and resuspended in neuron media. Unless noted otherwise, neuron media used in the instant example comprised MACS Neuro Media (Miltenyi Biotec, #130-093-570), 2 mM L-alanine-L-glutamine dipeptide (Sigma-Aldrich, G8541-100ML), and 1× B-27™ Plus Supplement (ThermoFisher Scientific, A3582801). Unless noted otherwise, cells were added onto PDL coated wells and placed inside a 5% CO2 incubator set at 37° C. for the stated days in vitro (DIV). Unless noted otherwise, 10,000 cells were plated per well with 0.056 cm² growth area.

### RT-qPCR for Determining Cell Culture Purity:

For the RT-qPCR assay, Directzol RNA micro-prep columns (Zymo, R2061) were used to extract RNA from neurons directly following neuron isolation. RNA concentration was measured using the Thermo Scientific™ NanoDrop 2000. Quantabio cDNA Synthesis kit (Quanta, 95047) was used to synthesize cDNA before conducting qPCR using the Quantabio PerfeCTa® SYBR® Green FastMix® (Quanta, 95073) on the ViiA7 Real Time PCR system (Life Technologies). The neuron enrichment in the negative fraction was calculated −ΔCT of ΔNeuN against ΔGFAP and ΔGLAST using the formula: −ΔCT=−(ΔCT NeuN−(SQRT(ΔCT GFAP²+ΔCT GLAST))). CT was calculated for each group based on the absolute CT per primer subtracted by the respective CT of the negative control to reduce background noise. For each group, RNA was extracted from 3 separate isolation procedures from young adult males and each sample was analyzed in triplicate. No outliers were detected nor omitted.

Primers used to identify the main cellular constituents included:

Neurons:
MAP2:
Forward:
(SEQ ID. NO: 1)
5′-CTG GAG GTG GTA ATG TGA AGA TTG-3′
Reverse:
(SEQ ID. NO: 2)
5′-TCT CAG CCC CGT GAT CTA CC-3′
NeuN:
Forward:
(SEQ ID. NO: 3)
5′ AAC CAG CAA CTC CACCCT TC-3′
Reverse:
(SEQ ID. NO: 4)
5′-CGA ATT GCC CGA ACA TTT GC-3′
Astrocytes:
GFAP:
Forward:
(SEQ ID. NO: 5)
5′-CTA ACG ACT ATC GCC GCC AA-3′
Reverse:
(SEQ ID. NO: 6)
5′-CAG GAA TGG TGA TGC GGT TT-3′
GLAST:
Forward:
(SEQ ID. NO: 7)
5′-CAA CGA AAC ACT TCT GGG CG-3′
Reverse:
(SEQ ID. NO: 8)
5′-CCA GAG GCG CAT ACC ACA TT-3′
Oligodendrocytes:
Oligo2:
Forward:
(SEQ ID. NO: 9)
5′-GAA CCC CGA AAG GTG TGG AT-3′
Reverse:
(SEQ ID. NO: 10)
5′-TTC CGA ATG TGA ATT AGA TTT GAG G-3′
β-actin:
Forward:
(SEQ ID. NO: 11)
5′-CTC TGG CTC CTA GCA CCA TGA AGA-3′
Reverse:
(SEQ ID. NO: 12)
5′-GTA AAA CGC AGC TCA GTA ACA GTC CG-3′

### Immunocytochemistry:

Cell cultures were fixed with 4% paraformaldehyde (PFA, 15 minutes) after the completion of the respective experiment. After fixation, immunocytochemistry was conducted by first washing the cells with DPBS 3 times, then incubating in 5% normal horse serum for 60 min to block nonspecific binding (VWR, 102643-676). Afterwards, the cells were incubated in 1:500 TUBB3 (BioLegend, 801202) for 16 hrs. followed by another 3 washes with DPBS and incubation in 1:500 Alexa Flour 488 (ThermoFisher Scientific, A32723) and 1:10000 DAPI (VWR, 95059-474) for 60 minutes, all conducted at room temperature. After the completion of immunocytochemistry, the cells were preserved in Fluoromount-G Mounting Medium (ThermoFisher Scientific, 00-4958-02) until imaging.

### Analysis of Neurite Outgrowth and Survival:

Representative images in the applicable figures were imaged using 20×/63× objectives on a Zeiss Axio Observer system. For automated image acquisition, the 20× magnification lens of the ImageXpress (IXM) Micro Confocal High-Content Imaging System (Molecular Devices, San Jose, CA) was used along with the Neurite Outgrowth Analysis Module in MetaXpress® 6 software (Molecular Devices) for automated image analysis, a system used to image and analyze changes in neuron morphology. Using the 20× magnification, 16 separate images (with 10% overlap) were required to sustain >90% coverage of each well while avoiding the walls.

Three variables were quantified:
Valid neurons: Total number of cells in a well that are both DAPI and TUBB3 positive and with total neurite outgrowth of ≥10 μm.
Total neurite outgrowth: Sum of the lengths of all the neurites from a valid neuron. This is then averaged over all the valid neurons in a well.
Average neurite length: The total length of all the neurites from a valid neuron divided by the number of neurites and branches of that cell. This is then averaged over all the valid neurons in the well.

### Compounds:

Compounds evaluated included RO48 (provided by Miami Project to Cure Paralysis, University of Miami), (S)-H-1152 (Cayman Chemical Company, 10007653), and 7-epi Paclitaxel (Cayman Chemical Company, 20741).

### Optimization of Surface Coating and Media Supplementation:

One analysis comprised if laminin coating, in addition to the PDL coated surface, would improve neurite outgrowth and number of valid neurons and/or mitigate the need for extra supplementation of media with B-27™ Plus (B27⁺). A 10 μg/mL laminin coating was applied for 1 hour at 37° C. in respective wells before being washed off with Dulbecco's Modified Eagle Medium (DMEM) prior to cell plating. Primary cortical neurons isolated from young adult male mice were cultured for 2DIV on PDL coated wells with (B27⁺) or without B27⁺ supplementation (Control), or on PDL/laminin coated plates with (B27⁺ & Laminin) or without B27⁺ supplementation (Laminin).

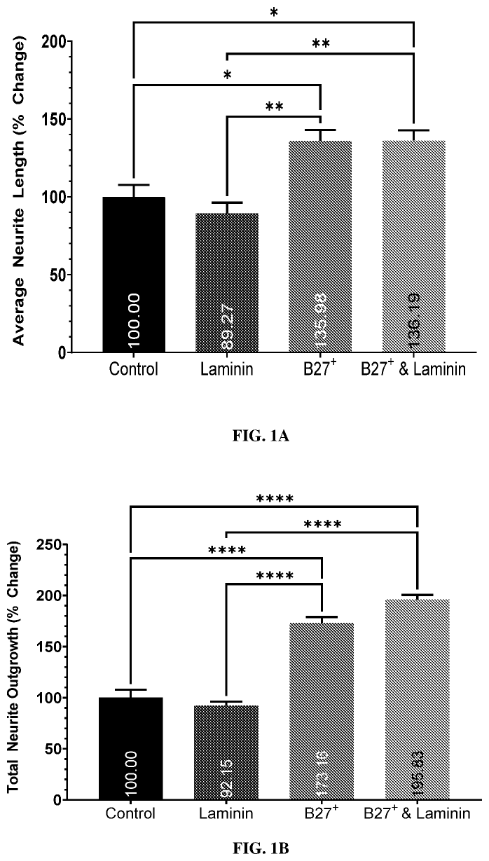

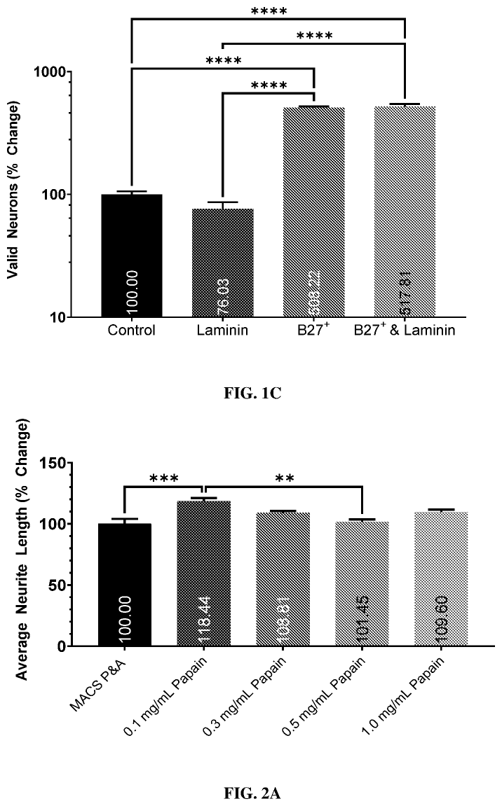

The average neurite length (FIG. 1A), total neurite outgrowth (FIG. 1B), and number of valid neurons (FIG. 1C) were analyzed and expressed as a percentage change relative to the control group containing no B27⁺ supplement or laminin coating. The B27⁺ supplementation induced a significant increase in average neurite length (P<0.05), total neurite outgrowth (P<0.0001), and number of valid neurons (P<0.0001). The extra laminin coating did not induce a significant changes with or without B27⁺ Plus supplementation. This suggests that for adult mouse cells, laminin did not improve neurite growth or number of valid neurons and cannot replace media supplementation.

Plating neurons without PDL coating resulted in few neurons adhering to the surface without substantial neurite growth. Media supplementation and surface coating increased the number of valid adult cortical neurons. For the instant example, PDL coating was used for subsequent experiments in standard size 384-well plates.

### Determine the Best Digestion Enzyme:

Another analysis determined the effectiveness of various digestion enzymes and concentrations to isolate cortical neurons and their efficacy to maintain their viability. Identical dissection methods, dissociation methods and temperatures, and volumes of digestive enzymes were utilized.

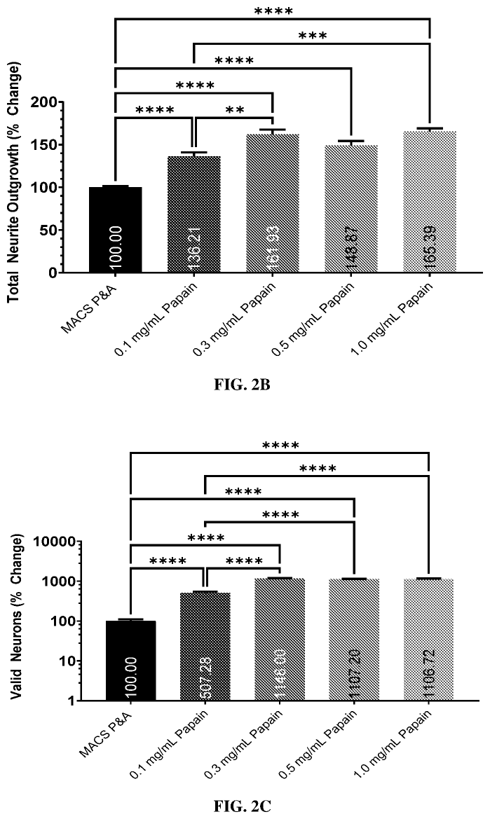

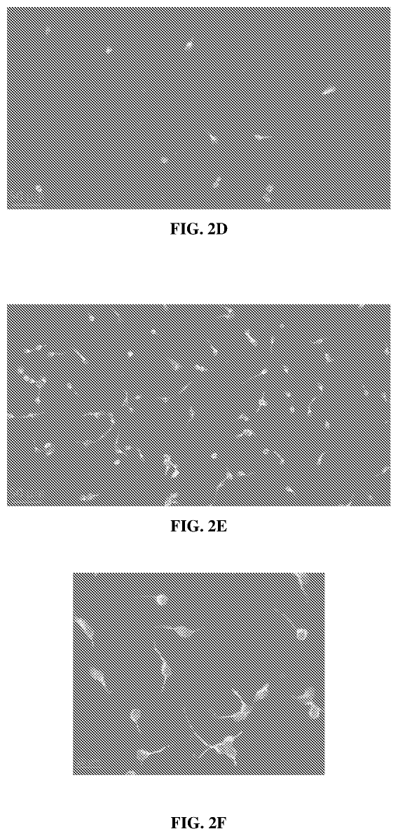

The respective digestive enzymes were used in replacement of 0.3 mg/mL papain to isolate cortical neurons from young adult male mice (1 mouse cortex per condition). Then the neurons were evenly distributed between 6 wells and cultured for 2DIV to measure how many cortical neurons can be extracted using each digestive enzyme and their ability to maintain neuron viability. The average neurite length, total neurite outgrowth, and number of valid neurons (FIGS. 2A-2C) were analyzed and expressed as a percentage change relative to the MACS® P&A enzymes included in the adult brain dissociation kit.

Then, 20× magnification images were taken of the cortical neurons isolated from young adult mice using MACS P&A (FIG. 2D) and 0.3 mg/mL papain (FIG. 2E). Overall, papain presented beneficial effects on cell survival and neurites outgrowth compared to the MACS® P&A digestive enzyme. A range of between 0.3-1.0 mg/mL papain resulted in increased total neurite outgrowth (P<0.0001) and number of valid neurons (P<0.0001) when compared to MACS® P&A. It was determined that 0.1 mg/mL papain had significantly higher total neurite outgrowth and number of valid neurons compared to MACS® P&A (P<0.0001), yet significantly lower values relative to higher papain concentrations (P<0.0001). This suggests that digestion with papain demonstrates a dose-dependent effect that plateaus at ≤0.3 mg/mL. Notably, 0.1 mg/mL papain had higher average neurite length compared to 0.5 mg/mL papain (P<0.01) and MACS® P&A (P<0.001), although still had similar total neurite outgrowth compared to 0.5 mg/mL papain. There were no significant differences between papain concentrations of 0.3-1.0 mg/mL for any analysis. Therefore, a concentration of 0.3 mg/mL (the lowest effective papain concentration), was used for subsequent experiments to reduce cost and potential off-target effects induced by higher concentrations. Accutase® and 0.25% Trypsin were also tested and yielded poorer outcomes compared to MACS® P&A.

### Evaluation of Dissociation Methods, Timings and Temperatures:

The effectiveness of different digestion methods, the incubation timing of those methods, and the incubation temperatures on the dissociation of cortical tissue from young adult male mice were determined. After cortical neurons are extracted with each respective protocol, the cells were evenly dispersed between 6 wells to analyze both the yield and viability of the extracted cortical neurons. The average neurite length, total neurite outgrowth, and number of valid neurons were expressed as a percentage change relative to the ABDK (30 minutes) group.

The gentleMACS Program 37C_ABDK_01 protocol, referred to as ABDK (30 minutes), is the standard protocol conducted at 37° C. using the gentleMACS™ Octo Dissociator with Heaters. To reduce the stress on cortical neurons, the duration of the protocol was shortened to 10 minutes and 20 minutes. To test the need and efficacy of the gentleMACS™ Octo Dissociator with Heaters, similar conditions were created by installing a revolving apparatus in the Thermo Scientific™ MaxQ™ 8000 Incubated Stackable Shakers that induced shaking of the contents inside.

Neurons dissociated using the gentleMACS™ Octo Dissociator with Heaters had longer average neurite lengths and total neurite outgrowth in comparison to neurons incubated in a revolving apparatus. The incubation time had no effect on neuron morphology when using gentleMACS™ Octo Dissociator with Heaters, yet, when using a rotating apparatus, a reduction in time lead to reduced total neurite growth (P<0.0001). The number of valid neurons was affected by method, timing, and temperature. Using the gentleMACS™ Octo Dissociator with Heaters increased the number of valid neurons by approximately 4-fold regardless of the incubation time relative to using a revolving apparatus at 37° C. (P<0.0001).

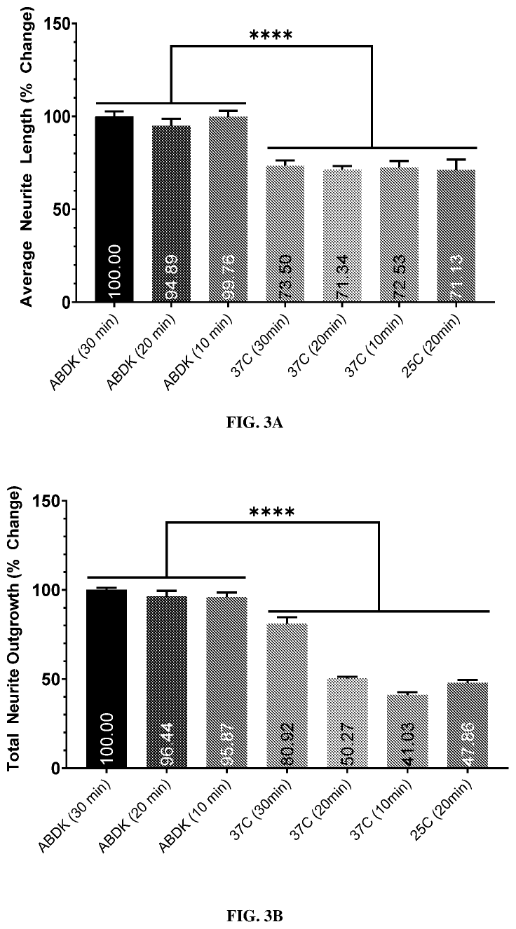

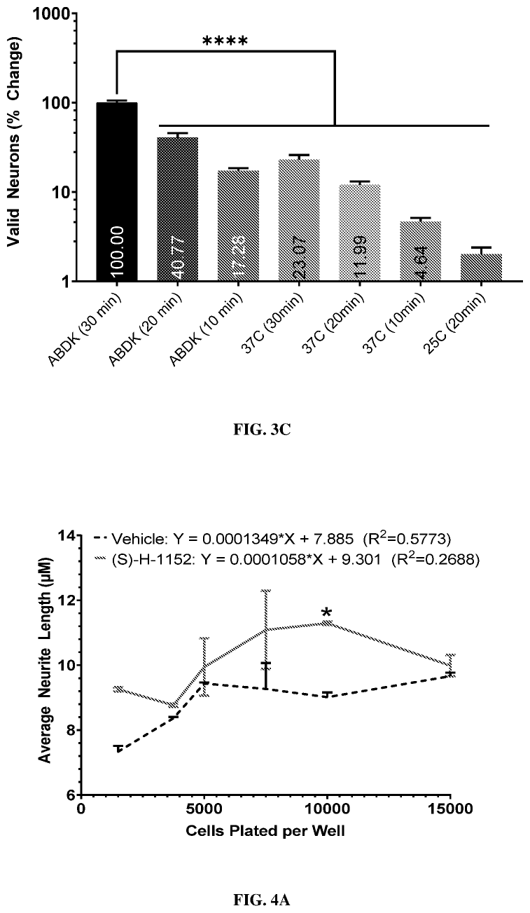

In each dissociation method, there was approximately a 2-fold increase in the number of valid neurons for every 10 minute increase in incubation time (P<0.0001). Furthermore, reducing the temperature from 37° C. to 25° C. during the 20 minute incubation in a revolving apparatus reduced the number of valid neurons without impacting neuron morphology (P<0.0001, FIG. 3C).

### Cell Plating Density:

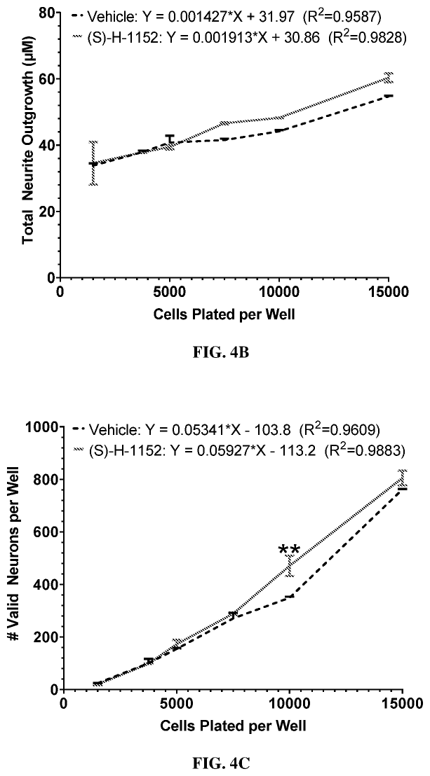

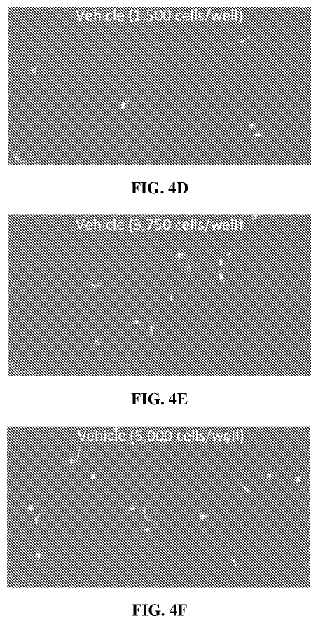

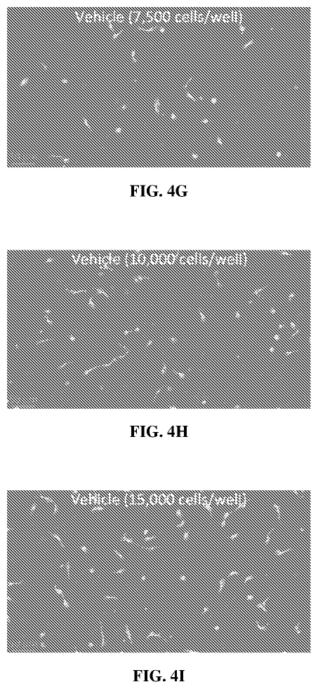

The reaction of neurons to compounds may be dependent on plating density. The effects of cell plating density of young adult cortical neurons were assessd on the average neurite length, total neurite outgrowth, and number of valid neurons per well (FIG. 4).

Cortical neurons were plated at 1,500, 3,750, 5,000, 7,500, 10,000, and 15,000 cells per well, in presence of (S)-H-1152 or Vehicle. (S)-H-1152 is a selective and potent rho-associated kinase (ROCK) inhibitor that attenuates KCl-induced contractions of femoral arteries and augments neurite outgrowth in dorsal root ganglion cells isolated from 1-day old rats that are cocultured with Schwan cells.

A simple linear regression analysis demonstrated no significant correlation between plating density and the average neurite outgrowth for both 0.05% DMSO (Vehicle) and 5 μM (S)-H-1152 treated groups. There was a significant positive correlation between both total neurite outgrowth (P values: Vehicle=0.0006, (S)-H-1152=0.0001) and number of valid neurons per well (P values: Vehicle=0.0006, (S)-H-1152<0.0001) and the cell plating density for both treatment groups. Only the slope of the total neurite outgrowth linear regression line differed significantly between treatment groups. In particular, (S)-H-1152 induced a steeper increase in total neurite outgrowth and therefore more responsive to increase plating density (P<0.05). Using two-way ANOVA to compare the means of different treatment groups at each respective plating density, (S)-H-1152 significantly increased the average neurite length (P<0.05) and number of valid neurons (P<0.01) when 10,000 cells were plated per well. It is suggested that plating density can be a considered for drug screenings. A concentration of 10,000 neurons per well was used in subsequent experiments.

### Efficacy of Various Neuronal Supplements:

To improve the survival of neurons and create an environment that resembles in vivo conditions, neuron supplements are added to media to study the synaptic function, neurite growth, and survival of primary neurons in vitro in a chemically defined manner without the use of serum. The efficacy of MACS® NeuroBrew®-21 (NeuroBrew, Miltenyi Biotec), B27⁺ (Gibco), and NeuroCult™ SM1 (SM1, Stemcell Technologies) as serum-free neuronal supplements to support neuron survival and neurite outgrowth were evaluated.

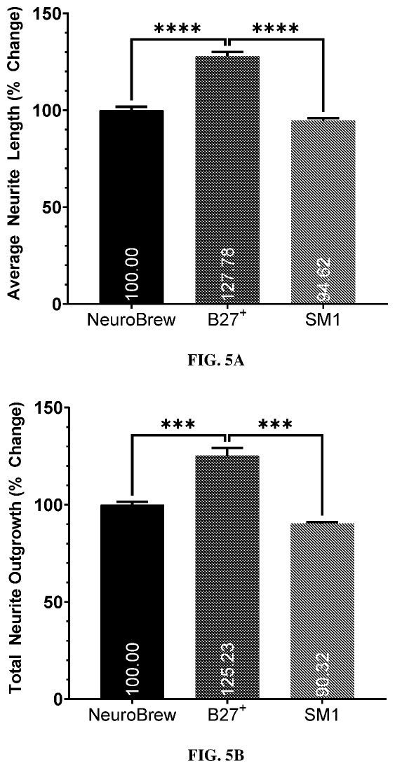

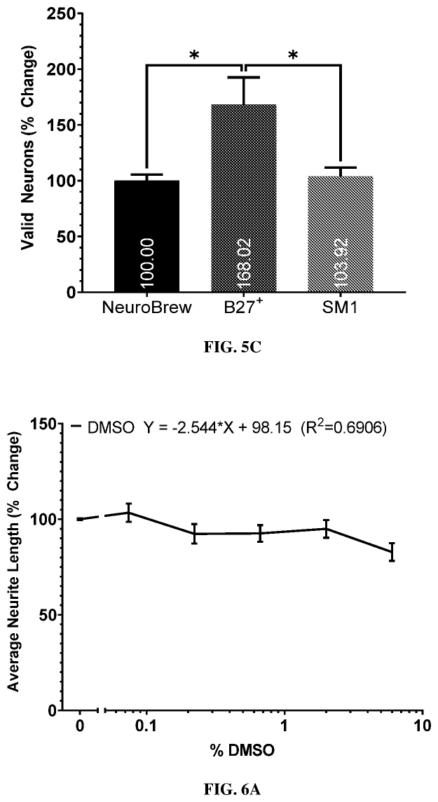

Primary cortical neurons from young adult male mice were plated at 10,000 cells per well and the effects of the different supplements on the average neurite length, total neurite outgrowth, and number of valid neurons were assessed as a percentage change relative to the NeuroBrew group (see FIG. 5). All supplements were used according to their respective instructional manuals and added to MACS® Neuro Medium. The use of B27⁺ resulted in an increase in the average neurite length (P<0.0001), total neurite outgrowth (P<0.001), and number of valid neurons (P<0.05) relative to all other cohorts. Therefore, B27⁺ can be used screenings to better nurture the adult neurons.

### The Effects of the Vehicle DMSO:

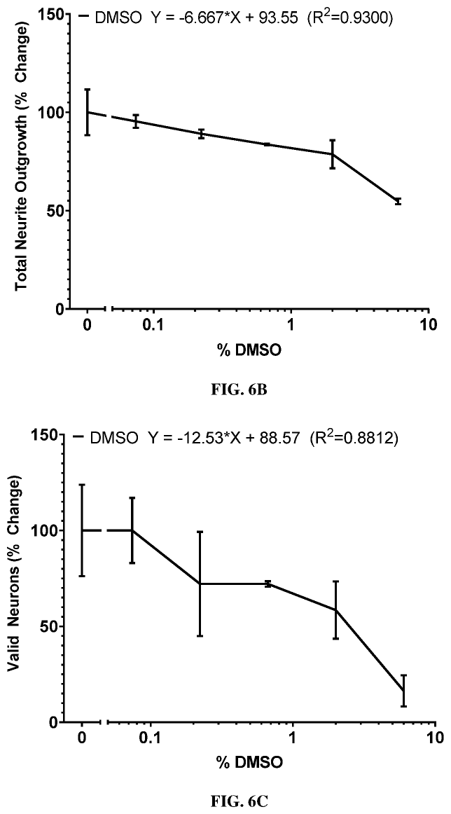

Dimethyl sulfoxide (DMSO) can be used to dissolve hydrophobic compounds and thus may be used as a vehicle in high content screenings. To determine the tolerance of adult neurons to DMSO, primary cortical neurons from young adult male mice were plated in the presence of different concentrations of DMSO and analyzed average neurite length, total neurite outgrowth, and number of valid neurons (FIG. 6). These factors were expressed as a percentage change relative to the negative control (0% DMSO). The linear regression analysis confirmed a significant negative correlation between average neurite length (P=0.0404), total neurite outgrowth (P=0.0019), and number of valid neurons per well (P=0.0055) and the percentage of the media containing DMSO (% DMSO). This suggests that DMSO concentrations should be minimized to avoid possible harmful effects on neurons.

Culture Purity Assessed with RNA Expression Analysis:

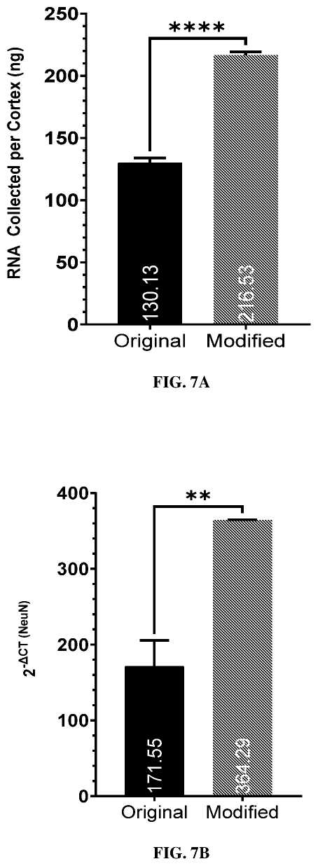

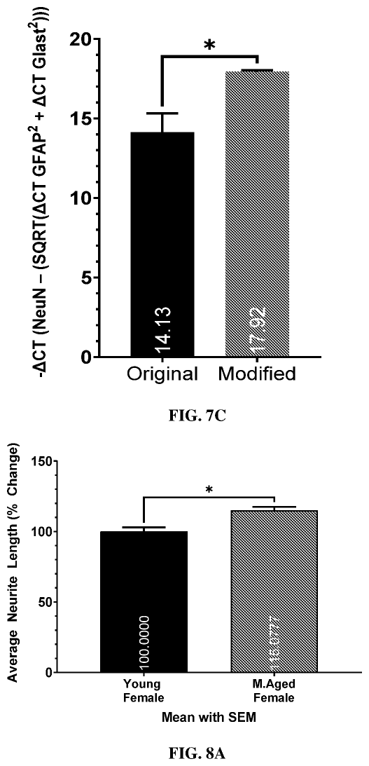

Protocol modifications described herein can provide culturing and screening of older adult cortical neurons. An RNA expression analysis was performed using RT-qPCR to determine how these modifications can affect the culture purity, in particular assessing RNA yield, neuron specific yield, and purity (FIG. 7).

The cells isolated from cortical tissue of young adult male mice were pelleted after the completion of the respective protocol followed by the extraction of RNA immediately thereafter. RT-qPCR data indicate that the modified protocol resulted in significantly more RNA being isolated from each cortex (P<0.0001), greater NeuN expression from the isolated cells (P<0.01), and increased NeuN expression relative to GFAP and GLAST (P<0.05). The in vitro cultures indicate that more cells are isolated and a larger percentage of these cells are neurons. The protocol of the instant example increased RNA yield by only 66% (FIG. 7A) yet increased the NeuN expression by 112% (FIG. 7B), suggesting an unexpectedly larger increase in neurons relative to other neural cells. This was confirmed by the significant increase in the relative NeuN expression in the protocol compared to the combination of GLAST and GFAP expression (FIG. 7C).

### Sex and Age-Dependent Effects of RO48 and Culturing Methods:

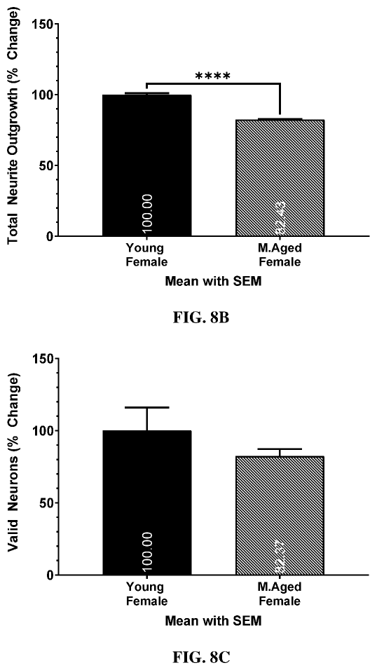

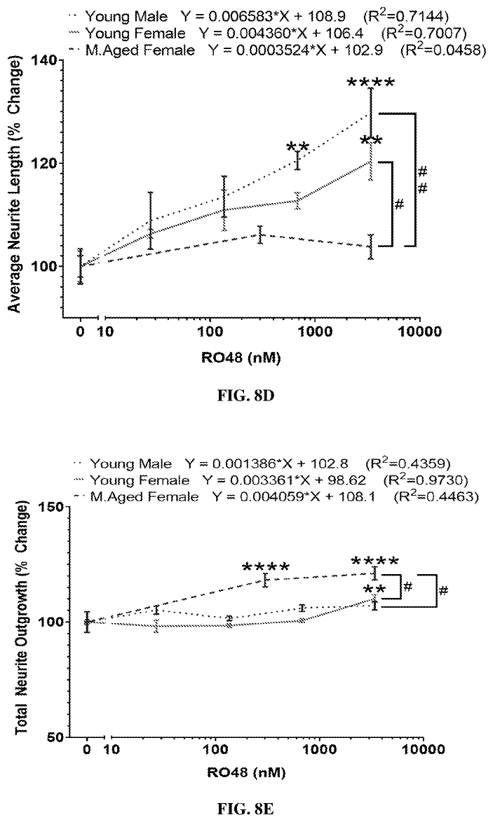

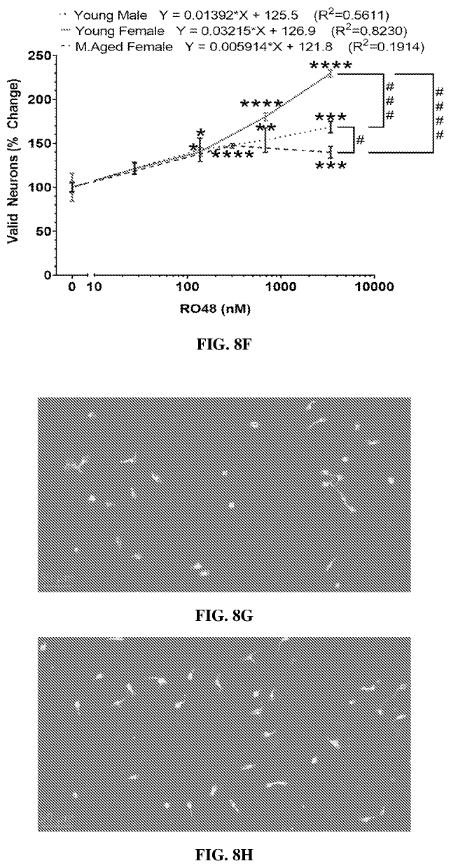

The effects of the isolation method, media, culturing protocol, and vehicle were analyzed for the young adult and middle-aged female cohorts. The middle-aged female cohort showed a small but significant increase in average neurite length and significant decrease in total neurite outgrowth compared to the younger female cohort (FIG. 8A-B). The middle-aged female cohort demonstrated a non-significant downward trend in number of valid neurons compared to the younger female cohort (FIG. 8C).

RO48 activates mammalian target of rapamycin complex (mTORC)1/2 and phosphatidylinositol-3-kinase (PI3K) and decreases the phosphorylation of S6-926. To determine its sex and age-dependent effects, RO48 was added to the media of adult neurons isolated from the cortex of young adult male, young adult female, and middle-aged female mice for 2DIV and average neurite length, total neurite outgrowth, and number of valid neurons were quantified (FIG. 8).

Thereafter, 20× magnification images were taken of primary cortical neurons isolated from middle-aged female mice treated with Vehicle (FIG. 8D) and 3400 nM RO48 (FIG. 8E) for 2DIV. A simple linear regression demonstrated a significant positive correlation between the RO48 concentration in the media and the total neurite outgrowth (P=0.0019) and number of valid neurons (P=0.0335) in the young adult female cohort only. Using a Šídák's multiple comparisons test, the means of each tested concentration of RO48 were compared to the cehicle treated group for each age cohort and to compare the relative percent change in value 3400 nM RO48 induced between the 3 age cohorts.

The average neurite length (FIG. 8D) increased more, relative to the vehicle, for the younger male and female cohorts compared to the middle-aged female cohort. The middle-aged female cohort was the only cohort without a significant increase in average neurite length at any concentration, resulting in a significant difference in the relative increase of neurite length at 3400 nM RO48 between younger cohorts. For the total neurite outgrowth (FIG. 8E), the middle-aged female cohort has a significant increase in neurite outgrowth compared to controls and significantly larger relative change compared to younger cohorts.

The young adult male and female cohorts do not differ significantly, although, the young adult female cohort does have a significant increase in neurite length at the highest dose. When analyzing the number of valid neurons (FIG. 8F), all three cohorts showed a significant increase in valid neurons at doses ≥300 nM, although the relative increase from the vehicle treatment differs significantly between the cohorts.

The young adult female cohort was shown to be most responsive, followed by the young adult male and middle-aged female cohort. These data demonstrate the sex- and age-dependent effects of RO48 on adult neurons in vitro which are more profound at higher RO48 concentrations. This suggests the importance of testing drug compounds in both sexes and age groups to determine demographic specific efficacies.

### The Age-Dependent Toxicity of 7-Epi Paclitaxel:

7-epi Paclitaxel is an FDA-approved drug for use in patients with ovarian cancer. 7-epi Paclitaxel stabilizes microtubule bundles, impairs organelle transport, induces peripheral neuropathy through the CXCR1/2 pathway, and reduces brain injury after repeated traumatic brain injuries in mice by inducing neurite growth and nerve regeneration.

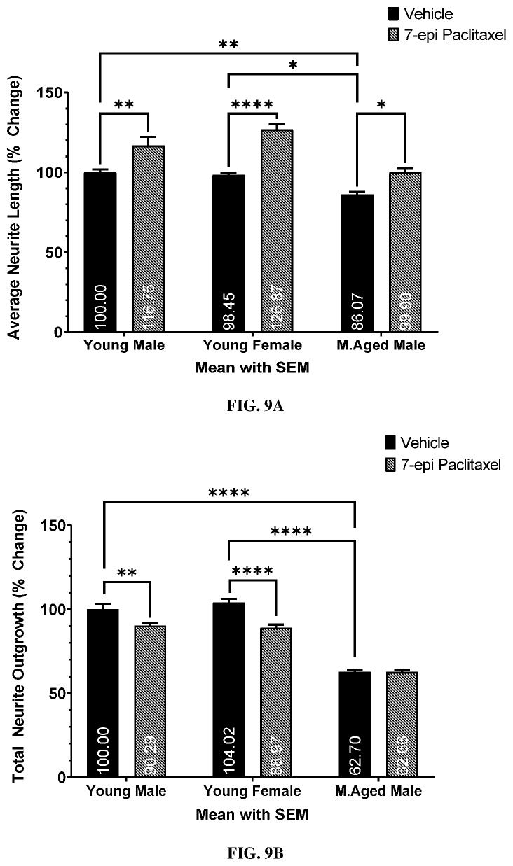

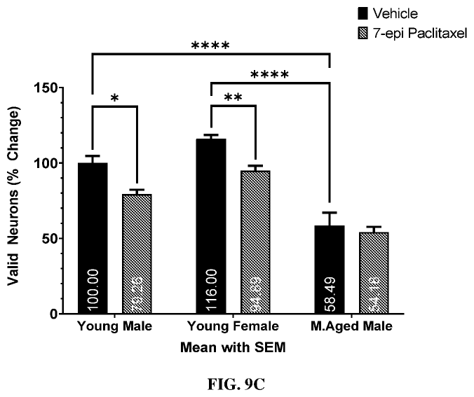

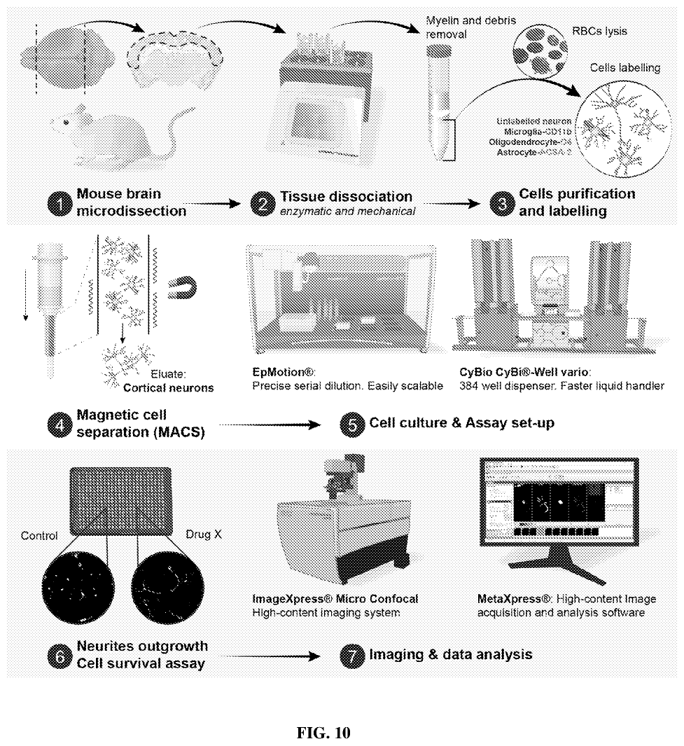

Adult neurons isolated from the cortex of young adult male, young adult female, and middle-aged male mice were treated with 150 nM 7-epi Paclitaxel, 3000 nM RO48, and Vehicle for 3DIV to analyze the average neurite length, total neurite outgrowth, and number of valid neurons (FIG. 9). One-way ANOVA with Dunnett's multiple comparisons test was used to compare the means of each compound to the mean of the Vehicle treated group within each respective cohort.

7-epi Paclitaxel significantly increased the average neurite length of all three age cohorts, yet only significantly decreased the total neurite outgrowth and number of valid neurons of the young adult cohorts without affecting the middle-aged male cohort.

This example demonstrates that screening 7-epi Paclitaxel in young adult neurons at 3DIV would yield an overall negative result and would have resulted in 7-epi Paclitaxel being prematurely dismissed from the assay. However, as demonstrated, 7-epi Paclitaxel yielded an overall positive result in middle-aged male neurons. This result suggests that studying the influence of 7-epi Paclitaxel in different cell demographics such as adult-aged celled is evidence of the importance of conducting age-appropriate screenings to mitigate premature dismissal of potentially beneficial compounds.

### Example 2

### Alternative Processing Method for Adult Mice Cortical Neurons

The instant example provides an alternative exemplary protocol for processing and culturing adult cortical neurons obtained from adult mice. The steps of the instant protocol are as follows:

1. Fill each of the wells on a 384-well glass bottom plate (MGB101-1-2-LG-L Brooks Life Sciences or 781892 Greiner-Bio 384 Well SensoPlate™) with 18 μL of 50 μg/mL Poly-D-Lysine (PDL), then tap plate on the side 5 times so liquid settles on glass surface.

2. Incubate plate in 5% CO2 incubator set at 37° C. for 1-48 hours

3. After incubation, wash the wells with H₂O 4× and dry at room temperature for 4-48 hours

4. After euthanizing mice, remove the brain from the head and place in cold Hank's balanced salt solution (HBSS) and microdissect region of interest

5. Fill each gentleMACS™ C Tube (Miltenyi Biotec, #130-093-237) with 5 mL of 0.3 mg/mL papain (Worthington LS003126) and place up to 1.25 grams of brain tissue in each gentleMACS™ C Tube

6. Cut the brain tissue in the gentleMACS™ C Tube into small pieces using scissors and quickly invert, make sure all brain pieces are in solution while tube is upside down

7. Place tube on the gentleMACS™ Octo Dissociator with Heaters (Miltenyi Biotec, #130-096-427) and attach the heating cuffs

8. Run the program “gentleMACS Program 37C_ABDK_01”

9. After program completion, strain the contents of the gentleMACS™ C Tube through a 70 μm cell strainer placed on top of a 50 mL conical centrifuge tube

10. Add 7 mL of cold Dulbecco's Phosphate-Buffered Saline with glucose and pyruvate (DPBS, Thermo Fisher Scientific, 14287072) into every gentleMACS™ C Tube used and add the contents into the respective 50 mL tube. Up to 5 grams of digested tissue can be strained into each 50 mL tube

11. Centrifuge the 50 mL tubes at 300×g for 7 minutes at 4° C. and aspirate supernatant completely afterwards

12. Prepare Debris Removal Solution: 1800 μL of Debris Removal Concentrate (Miltenyi Biotec, #130-109-398) mixed in 6200 μL cold DPBS

13. Resuspend pellet in 8 mL Debris Removal Solution for every 1.25 grams of brain tissue

14. Mix contents of tube until sample is resuspended and solution appears homogenous

15. Very slowly, add 4 mL of cold DPBS on top of the solution. DPBS should be visibly settling on top, not mixed with the homogenous solution

16. Centrifuge at 4° C. and 3000×g for 5 minutes with slow acceleration and deceleration

17. Aspirate out all of the top layer of clear liquid and any flat layers of myelin, leave the debris below the flat layer of myelin in the tube and minimize the amount of debris removal solution aspirated

18. Fill DPBS until the 50 mL mark

19. Centrifuge the 50 mL tubes at 300×g for 5 minutes at 4° C. and aspirate supernatant completely afterwards

20. Prepare Red Blood Cell Remover Solution: 125 μL Red Blood Cell Lysis Solution 10× (Miltenyi Biotec, #130-094-183) mixed with 1125 μL of H₂O

21. Resuspend each pellet in 1.25 mL of Red Blood Cell Remover Solution for every 1.25 grams of starting brain tissue

22. Incubate the tubes in the dark at 4° C. for 10 minutes

23. Make 0.5% bovine serum albumin (BSA) solution: 25 mL of MACS BSA Stock Solution (Miltenyi Biotec, #130-091-376) mixed in 475 mL DPBS

24. Add 12.5 mL of 0.5% BSA for every 1.25 grams of starting brain tissue

25. Centrifuge at 300×g for 5 minutes at 4° C. and aspirate supernatant completely afterwards

26. Resuspend each pellet in 80 μL of 0.5% BSA and 20 μL of Non-Neuronal Cells Biotin-Antibody Cocktail (Miltenyi Biotec, #130-115-389) for every 1.25 grams of starting brain tissue and transfer solution into 15 mL conical centrifuge tubes

27. Mix well, then incubate in the dark for 5 minutes at 4° C.

28. Add 1 mL of 0.5% BSA for every 1.25 grams of cortical tissue

29. Centrifuge at 300×g for 5 minutes at 4° C. and aspirate supernatant completely afterwards

30. Resuspend each pellet in 80 μL of 0.5% BSA and 20 μL of Anti-Biotin MicroBeads (Miltenyi Biotec, #130-115-389)

31. Mix well, then incubate in the dark for 10 minutes at 4° C.

32. During this time, wash the LS column (Miltenyi Biotec, #130-042-401) by adding 5 mL of 0.5% BSA as it is attached to QuadroMACS™ Separator (Miltenyi Biotec, #130-090-976)

33. Add 5 mL of 0.5% BSA into the 15 mL tubes after incubation time and mix gently

34. Transfer all liquid in 15 mL tube into the washed LS columns and collect liquid being expelled (using only the force of gravity) as the negative fraction (neurons)

35. Place the LS columns on new 15 mL tubes and add 5 mL of 0.5% BSA into LS column

36. Use the included plunger to quickly force the liquid inside the LS column into the 15 mL tube and collect as positive fraction (non-neuron neural cells)

37. Centrifuge the 15 mL tubes at 300×g for 5 minutes at 4° C. to pellet and resuspend neurons in 30 μL of neuron media. The neuron media comprises MACS Neuro Media (Miltenyi Biotec, #130-093-570); 2 mM L-alanine-L-glutamine dipeptide (Sigma-Aldrich, G8541-100ML); 1× B-27™ Plus Supplement (ThermoFisher Scientific, A3582801); optionally 50 units/mL of penicillin and 50 μg/mL of streptomycin (Corning, 30-002-CI), optionally ≤0.075% DMSO

38. Add neurons into each of the PDL coated wells (˜10,000 neurons in each well)

39. Place cells in 5% CO2 for the stated days in vitro (DIV)

### Example 3

### Processing and Screening Method for Adult Large Animal Neurons

According to the instant example, protocols, methods, and applications of processing and screening using adult neurons obtained from adult large animals can be utilized. The processes and methods of the examples described herein can be adapted for use in various large animals. For instance, when animals other than mice are used as the source for neural cells, the steps regarding Magnetic Cell Separation (MACS) may be omitted from the methods as they may not be necessary for the particular animal.

Large animals that can be used according to the instant example include but are not limited to sheep, cows, pigs, horses, goats, and the like. It is contemplated that any mammal can be utilized for the instant example, including livestock mammals. FIG. 11 shows an exemplary processing and screening method using neural cells obtained from sheep.

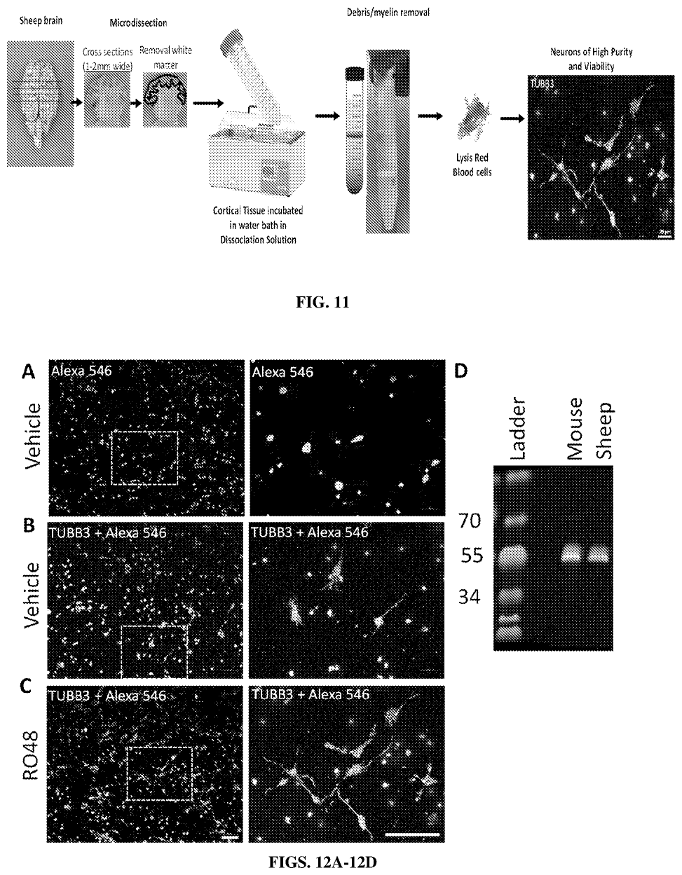

FIGS. 12A-12D demonstrate a screen of cortical neurons obtained from an adult sheep brain that was 2 years old. The cortical neurons were cultured for 2DIV and stained with secondary antibody Alexa 456 (Goat) only (FIG. 12A), or with TUBB3 primary antibody with Alexa 546 (FIGS. 12B and 12C). Cells were treated with either vehicle control (FIGS. 12A-12B) or with 2.5 μM of RO48 treatment (FIG. 12C). Further, in FIG. 12D, a TUBB3 Western blot is shown against 15 μg of protein from mouse and sheep cortices next to iBright ladder.

Furthermore, the processes and methods of the examples described herein can be adapted for use of the instant example using any cell type in the central nervous system (CNS), including all regions of the brain and also all spinal regions (e.g., spinal cord).

1. A method of processing neural cells, said method comprising the steps of:
extracting one or more brain components from an animal,
dissociating cells from the brain components to form a brain cell composition,
purifying the neural cells from the brain cell composition, and
culturing the neural cells.

2. The method of claim 1, wherein the disassociating step comprises enzymatic disassociation, mechanical disassociation, or both.

3. The method of claim 1, wherein the purifying step comprises removal of myelin from the brain cell composition.

4. The method of claim 1, wherein the purifying step comprises removal of debris from the brain cell composition.

5. The method of claim 1, wherein the purifying step comprises removal of red blood cells from the brain cell composition.

6. The method of claim 1, wherein the purifying step comprises separation of the neural cells from glial cells.

7. The method of claim 1, wherein the purifying step comprises use of papain.

8. The method of claim 1, wherein the neural cells comprise primary cells.

9. (canceled)

10. The method of claim 1, wherein the neural cells comprise adult animal cells, wherein the adult animal cells are young adult animal cells.

11. The method of claim 1, wherein the neural cells comprise adult animal cells, wherein the adult animal cells are middle-aged adult animal cells.

12. The method of claim 1, wherein the neural cells comprise adult animal cells, wherein the adult animal cells are senior adult animal cells.

13. (canceled)

14. (canceled)

15. (canceled)

16. (canceled)

17. (canceled)

18. (canceled)

19. (canceled)

20. (canceled)

21. The method of claim 1, wherein the neural cells comprise cortical cells.

22. The method of claim 1, wherein the neural cells comprise cortical astrocyte cells.

23. The method of claim 1, wherein the neural cells comprise cortical neuron cells.

24. The method of claim 1, wherein the neural cells comprise spinal cells.

25. The method of claim 1, wherein the neural cells comprise spinal astrocyte cells.

26. The method of claim 1, wherein the neural cells comprise spinal neuron cells.

27. The method of claim 1, wherein the neural cells comprise hippocampal cells.

28. The method of claim 1, wherein the neural cells comprise hippocampal astrocyte cells.

29. The method of claim 1, wherein the neural cells comprise hippocampal neuron cells.

30-58. (canceled)
